(19) United States
(12) Patent Application Publication
(10) Pub. No.: US 2022/0387556 A1
(43) Pub. Date: Dec. 8, 2022

(54) USE OF THE GDF-5 MUTANT FOR THE TREATMENT OF PAIN AND CARTILAGE DESTRUCTION

(71) Applicant: Merck Patent GmbH, Darmstadt (DE)

(72) Inventors: Kerstin KLEINSCHMIDT-DOERR, Darmstadt (DE); Anne GIGOUT, Darmstadt (DE); Daniela WERKMANN, Darmstadt (DE)

(73) Assignee: Merck Patent GmbH, Darmstadt (DE)

(21) Appl. No.: 17/783,704

(22) PCT Filed: Dec. 15, 2020
(86) PCT No.: PCT/EP2020/086145

§ 371 Date: Jun. 9, 2022

(30) Foreign Application Priority Data
Dec. 18, 2019 (EP) 19217389.6

(51) Int. Cl. A61K 38/18; A61K 47/42; A61P 19/02

(52) U.S. Cl. CPC A61K 38/1825; A61K 47/42; A61P 19/02

(57) ABSTRACT

The present invention is directed to the use of the GDF-5 mutant with an amino acid exchange R399E for the treatment of cartilage defects and pain and a pharmacological composition of said GDF-5 mutant.

### BACKGROUND OF THE INVENTION

### Field of the Invention

The present application relates to the use of the GDF-5 mutant (R399E), formulations thereof and therapeutic compositions for injection in patients suffering from Osteoarthritis (OA) or other inflammatory join diseases for the reduction of pain and inflammation.

Osteoarthritis is the most common form of arthritis, affecting millions of people worldwide. It occurs when the protective cartilage that cushions the ends of bones wears down over time. Although osteoarthritis can damage any joint, the disorder most commonly affects joints in hands, knees, hips and spine.

The rupture of cartilage homeostasis whatever its cause (aging, genetic predisposition, trauma or metabolic disorder) induces profound phenotypic modifications of chondrocytes, which then promote the synthesis of a subset of factors that induce cartilage damage and target other joint tissues. Interestingly, among these factors are numerous components of the inflammatory pathways. Chondrocytes produce cytokines, chemokines, alarmins, prostanoids, and adipokines and express numerous cell surface receptors for cytokines and chemokines, as well as Toll-like receptors. These receptors activate intracellular signaling pathways involved in inflammatory and stress responses of chondrocytes in OA joints (Houard et al. Curr. Reumathol Rep., 2013, November; 15(11):375).

OA symptoms can only be managed by pain drugs for a period, but the damage to joints can't be reversed. Staying active and maintaining a healthy weight might slow progression of the disease and help improve pain and joint function. Nevertheless, there is no effective treatment available to stop or even reverse the disease and, in most cases, both, destruction of the affected joint and pain progress and significantly impact the patient's mobility, quality of life and ability to work. Total joint replacement surgery is often the unavoidable and only treatment option for patients with hip or knee OA, while pain management is often the only option for other joints.

A recent publication showed that radiographic knee OA was associated with a higher risk of cardiovascular diseases, diabetes and renal mortality, especially in people with early onset of the disease or with obesity (Mendy et al., Int. J. Epidemiol. 2018, Dec. 1; 47(6):1821).

In 2015, 10.5% (25.6 million) of noninstitutionalized US adults reported having OA. Adults with OA incurred $318.4 billion in healthcare costs, representing 22.5% of the total healthcare costs of US noninstitutionalized adults, and suffered $10.1 billion in lost wages. OA prevalence varied significantly by age (≥65 years, 25.3%), sex (female, 13.3%; male, 7.5%), and race/ethnicity (White, 13.3%; African-American, 7.5%; Latino, 4.2%; other, 5.3%; P<0.001). Almost one-third of adults with OA (32.7%) received prescription opioids vs 13.8% of those without OA (P<0.001). Regression analyses indicated that adults with OA were significantly more likely than those without OA to report moderate (adjusted odds ratio [AOR]=1.99 [95% Cl: 1.65-2.40]) or severe (AOR=2.59 [2.21-3.04]) PIA, any functional limitation (AOR=2.51 [2.21-2.85]), and poorer HRQoL on the SF-12 Physical Component Summary (adjusted beta=−3.88 [SE: 0.357]; P<0.001). Adjusted incremental annual total healthcare costs and lost wages among adults with OA relative to those without OA were $1778 ($7585 vs $5807) and $189 ($740 vs $551) per person, respectively, resulting in an estimated national incremental direct cost of $45 billion and indirect cost of $1.7 billion (DOI: https://doi.org/10.1016/j.jval.2018.04.012).

Currently there is no Osteoarthritis treatment available that has both, a beneficial effect on tissue structure pathology (cartilage, bone, synovial membrane, meniscus, ligaments) and rapid relieving effect on pain, neither acute nor chronic.

GDF-5 (Hotten et al. 1994, Biochem. Biophys Res. Commun. 204, 646-652, NCBI Acc. No. NM_000557, NP_000548) is a morphogen which has been shown to promote cell proliferation, differentiation and/or tissue formation in several tissues. The protein is also known as bone morphogenetic protein-14 (BMP-14) or cartilage-derived morphogenetic protein-1 (CDMP-1). GDF-5 shows chondrogenic activity and congenital GDF-5 mutations cause defects in digit, wrist and ankle joints in mice and humans (Storm et al., 1994; Thomas et al., 1997). The expression of GDF-5 is most strikingly limited to regions where joints will develop and is one of the earliest markers of joint formation (Storm and Kingsley, 1999). BMP receptor signaling is required for postnatal maintenance of articular cartilage (Rountree, 2004, PLoS Biol. 2004 November, 2(1)

Wildtype GDF5 treatment induces the formation of cartilage and bone. Therefore, a GDF5 single point mutant was designed in which the amino acid residue 399 arginine was exchange by glutamic acid. In the following named R399E. R399E shows a reduced bone formation potential compared to GDF5 wildtype with a sustained chondrogenic potential. R399E (GDF5 mutant) increases matrix production in primary porcine and human osteoarthritic chondrocytes (T. Mang, K. Kleinschmidt-Doerr, F. Ploeger, S. Lindemann, A. Gigout, DOI: https://doi.org/10.1016/j.joca.2018.02.176. April 2018 Volume 26, Supplement 1, Page S82).

R399E is claimed in WO2013083649A1 and has an improved capability of inducing cartilage formation. The recombinant GDF-5 related proteins of that invention are particularly suitable for use in the treatment of diseases, wherein the formation of cartilage is desired, but the formation of bone is undesirable. Thus, another aspect of that invention is the use of the named proteins, nucleic acids, vectors or host cells in the treatment of these diseases. In particular, the proteins, nucleic acids, vectors or host cells are for use in the treatment of cartilage defects or for the treatment of traumatic rupture or detachment of cartilage, including osteoarthritis.

Field of GDF-5

GDF-5 related proteins having an improved capability of inducing cartilage formation and a reduced capability of inducing bone formation. The novel proteins are particularly useful in the treatment of cartilage defects, wherein the formation of bone tissue is undesirable.

Synovial joints are essential for the biomechanical function of the skeleton. An improper function as observed in arthritic diseases directly results in a severe loss of life quality. Therefore, joint biology has been in focus of extensive research for years leading to an understanding of joint anatomy and histology as well as the biomechanical properties and roles of articular cartilage and other components in joint function and maintenance.

GDF-5 (Hoetten et al. 1994, Biochem. Biophys Res. Commun. 204, 646-652) is a morphogen which has been shown to promote cell proliferation, differentiation and/or tissue formation in several tissues. The protein is also known as morphogenic protein MP52, bone morphogenetic protein-14 (BMP-14) or cartilage-derived morphogenetic protein-1 (CDMP-1). GDF-5 shows chondrogenic activity and congenital GDF-5 mutations cause defects in digit, wrist and ankle joints in mice and humans (Storm et al., 1994; Thomas et al., 1997). The expression of GDF-5 is most strikingly limited to regions where joints will develop and is one of the earliest markers of joint formation (Storm and Kingsley, 1999). BMP receptor signaling is required for postnatal maintenance of articular cartilage (Rountree, 2004, PLoS Biol. 2004 November, 2(11) GDF-5 is closely related to GDF-6 and GDF-7. These three proteins form a distinct subgroup of the TGF-beta superfamily, thus displaying comparable biological properties and an extraordinary high degree of amino acid sequence identity (see i.e. Wolfman et al. 1997, J. Clin. Invest. 100, 321-330). All family members are initially synthesized as larger precursor proteins which subsequently undergo proteolytic cleavage at a cluster of basic residues approximately 110-140 amino acids from the C-terminus, thus releasing the C-terminal mature protein parts from the N-terminal pro-domain. The mature polypeptides are structurally related and contain a conserved bioactive domain comprising six or seven canonical cysteine residues which is responsible for the characteristically three-dimensional “cystine-knot” motif of these proteins. Native GDF-5 related proteins are homo-dimeric molecules and act mainly through interaction with specific receptor complexes which are composed of type I and type II serine/threonine receptor kinases. The receptor kinases subsequently activate Smad proteins, which then propagate the signals into the nucleus to regulate target gene expression.

It has repeatedly been demonstrated that members of the GDF-5/-6/-7 subgroup are primarily important inducers and regulators of bone and cartilage (Cheng et al. 2003, J. Bone and Joint Surg. 85A, 1544-1552; Settle et al. 2003, Developm. Biol. 254, 116-130). GDF-5 and related proteins bind to and oligomerize two types of membrane bound serine-threonine kinase receptors termed type I and II. Upon ligand binding, these complexes transduce signals by phosphorylating members of the SMAD family of transcription factors, which upon activation enter the nucleus and regulate transcription of responsive genes (Massague, 1996). Recent experiments have implicated two different type I receptors in skeletal patterning, BMPR-IA and BMPR-IB. Both receptors are expressed in dynamic patterns during normal development. In several limb structures, for example, in joint interzones and perichondrium, an overlapping expression of BMPR-IA and BMPR-IB is observed (Mishina et al., 1995; Zou et al, 1997; Baur et al, 2000). With regard to the BMPR-IA and BMPR-IB expression patterns, GDF-5 signal transduction should be accomplished by the interaction with both BMPR-IA and BMPR-IB (Chang et al., 1994; Zou et al., 1997). Null mutations in the bmpr-1b gene produce viable mice with defects in bone and joint formation that closely resemble those seen in mice missing GDF-5 (Storm and Kingsley, 1996; Yi et al, 2000), whereas bmpr-Ia/mice are known to die early in embryogenesis (Mishina et al, 1995). However, a conditional knockout of BMPR-IA under the control of a GDF-5-Cre driver bypasses embryonic lethality and produces viable mice with normally formed joints. But, after birth articular cartilage within the joints wears away in a process reminiscent to osteoarthritis, which points at the importance of this receptor in cartilage homoeostasis and repair (Rountree et al., 2004).

The activity of the wild-type proteins of GDF-5 related protein family generally results in the formation of cartilage and bone. However, different medical conditions exist, wherein a formation of cartilage is desirable, however, the formation of bone tissue is undesired. For example, it is evident that in case of joint defects, the formation of cartilage is desirable whereas ossification should be avoided.

Surprisingly, it was found out that it is possible to provide variants of GDF-5 related proteins having an improved capability of inducing cartilage formation and a reduced capability of inducing bone formation. This can be achieved by modifying GDF-5 related protein (R399E) such that they have an increased affinity for the BMPR-IB and/or a reduced affinity for the BMPR-IA and is subject matter of WO2013083649A1 the closest prior art.

Wild-type GDF-5 binds BMPR-IB in vitro with about 40- to 120-fold higher affinity (KD ˜8-27 pM) as compared with BMPR-IA (KD ˜1-1.1 nM). It was found that by modifying the binding affinity of GDF-5 related proteins such that the affinity for BMPR-IB is increased while the affinity for BMPR-IA is reduced, cartilage formation is facilitated while the formation of bone is reduced. This can be achieved by specific substitutions of one or more amino acid residues relating to a BMPR-IB and/or BMPR-IA binding site in the amino acid sequence of a GDF-5 related protein.

The binding affinity of GDF-5 related proteins having specific substitutions is compared to the binding affinity of human wild-type GDF-5 related protein, in particular human wild-type GDF-5.

In order to avoid misunderstandings and ambiguities, some frequently used terms herein are defined and exemplified as follows: The term “cystine-knot domain” as used herein means the well-known and conserved cysteine-rich amino acid region which is present in the mature parts of TGF-beta superfamily proteins such as i.e. human GDF-5 and forms a three-dimensional protein structure known as cystine-knot. In this domain the respective location of the cysteine residues to each other is important and is only allowed to vary slightly in order not to lose the biological activity. It has been demonstrated that the cystine-knot domain alone is sufficient for the biological function of the protein (Schreuder et al. (2005), Biochem Biophys Res Commun. 329, 1076-86). Consensus sequences for cystine-knot domains are well known in the state of the art. According to the definition defined herein the cystine-knot-domain of a protein starts with the first cysteine residue participating in the cystine-knot of the respective protein and ends with the residue which follows the last cysteine participating in the cystine-knot of the respective protein.

The term “GDF-5-related protein” as used herein means any naturally occurring or artificially created protein which is very closely related to human growth/differentiation factor 5 (hGDF-5). Common feature of all GFD-5-related proteins is the occurrence of a cystine-knot-domain with an amino acid identity of at least 60 percent to the 102 aa cystine-knot domain of human GDF-5 (amino acids 400-501), which is sufficient for the biological function of the protein. The term “GDF-5-related proteins” includes proteins belonging to the group of GDF-5, GDF-6 and GDF-7 proteins from vertebrate or mammalian species as well as recombinant variants thereof as long as these proteins show the above-mentioned percentage of identity with the cystine-knot domain of human GDF-5. The limiting value of 60 percent is well suitable to separate members of the GDF-5/-6/-7 group of proteins as well as variants thereof from further proteins such as more distantly related GDFs and BMPs. A comparison of the 102 aa cystine-knot-domains of human GDF-5, human GDF-6 and human GDF-7 reveals the high grade of amino acid identity between these proteins. Human GDF-6 shares 87 (85 percent) and human GDF-7 shares 83 (81 percent) identical residues with the cystine-knot-domain of human GDF-5. The respective domains of GDF-5/-6/-7 molecules from other vertebrate and mammalian species which have been identified so far also show very high identity percentages of at least 75 percent (between 79 percent and 99 percent), when compared with human GDF-5. In contrast, GDFs and BMPs not belonging to the GDF-5/-6/-7 subgroup display much lower identity values below 60 percent.

Non-limiting examples for vertebrate and mammalian GDF-5-related proteins are precursors and mature proteins of human GDF-5 (disclosed as MP52 in WO95/04819 and as human GDF-5 in Hotten et al. 1994, Biochem. Biophys Res. Commun. 204, 646-652), recombinant human (rh) GDF-5/MP52 (WO96/33215), MP52 Arg (WO97/06254); HMW human MP52s (WO97/04095), CDMP-1 (WO96/14335), mouse (Mus musculus) GDF-5 (U.S. Pat. No. 5,801,014), rabbit (Oryctolagus cuniculus) GDF-5 (Sanyal et al. 2000, Mol Biotechnol. 16, 203-210), chicken (Gallus gallus) GDF-5 (NCBI accession no. NP_989669), african clawed frog (Xenopus laevis) GDF-5 (NCBI accession no. AAT99303), monomeric GDF-5 (WO 01/11041 and WO 99/61611), human GDF-6/BMP-13 (U.S. Pat. No. 5,658,882), mouse GDF-6 (NCBI accession no NP_038554), GDF-6/CDMP-2 (WO96/14335), human GDF-7/BMP-12 (U.S. Pat. No. 5,658,882), mouse GDF-7 (NCBI accession no AAP97721), GDF-7/CDMP-3 (WO96/143335). Covered by the invention are also GDF-5-related proteins having additional mutations such as substitutions, additions and deletions, as long as these additional mutations do not completely abolish the biological protein activity.

### Discussion of Background of the Invention

There is no drug available to treat both, pain and inflammation rapidly and lasting, and structural changes of joint tissue and morphology (cartilage, bone, synovial membrane, meniscus, ligaments) sustainably at the same time in OA patients.

Medications that can help relieve osteoarthritis symptoms and primarily pain, but have no or even negative effect on structures include:

Acetaminophen. Acetaminophen (Tylenol, others) has been shown to help some people with osteoarthritis who have mild to moderate pain. Taking more than the recommended dose of acetaminophen can cause liver damage.

Nonsteroidal anti-inflammatory drugs (NSAIDs). Over-the-counter NSAIDs, such as ibuprofen (Advil, Motrin IB, others) and naproxen sodium (Aleve, others), taken at the recommended doses, typically relieve osteoarthritis pain. Stronger NSAIDs are available by prescription. NSAIDs can cause stomach upset, cardiovascular problems, bleeding problems, and liver and kidney damage. NSAIDs as gels, applied to the skin over the affected joint, have fewer side effects and may relieve pain just as well.

Duloxetine (Cymbalta). Normally used as an antidepressant, this medication is also approved to treat chronic pain, including osteoarthritis pain.

Cortisone injections. Injections of corticosteroid medications may relieve pain in the joint. The number of cortisone injections patients can receive each year is generally limited to three or four injections, because the medication can worsen joint damage over time.

An anti-NGF (nerve growth factor) antibody (tanezumab, Pfizer) is currently under clinical development for OA. The compound is highly efficient to treat pain in OA patients, but has, like other pain drugs no beneficial effects on the underlying cause of the disease. In contrast, in a meaningful number of patients treated with anti-NGF, the disease progression was significantly accelerated (RPOA=rapidly progressing OA). The incidence of RPOA overall was 6.3 percent in the tanezumab 5 mg arm, 3.2 percent in the tanezumab 2.5 mg arm and 1.2 percent in the NSAIDs arm. We confirmed these negative effects of anti-NGF treatment in animal models of OA in rats and rabbits.

Pain drugs available to treat OA pain have significant side effects and adverse events. None of them slows down or halters disease progression or has beneficial effects on joint structures. None of them has healing or beneficial biological activity on cartilage matrix production or is re-balancing the pathological shift of joint homeostasis. Some (tanezumab) even accelerated disease progression in OA patients.

Other available OA therapies are surgical procedures or have no disease modifying effect:

Lubrication injections. Injections of hyaluronic acid may offer pain relief by providing some cushioning in the knee, though research suggests these injections offer no more relief than a placebo and have no effect on structural changes and tissue pathology at all.

Joint replacement. In joint replacement surgery (arthroplasty), the surgeon removes the damaged joint surfaces and replaces them with plastic and metal parts. Surgical risks include infections and blood clots. Artificial joints can wear out or come loose and may need to eventually be replaced.

There is one structure modifier under clinical development for OA, but this molecule does not have beneficial effects on OA pain. Fibroblast growth factor 18 (FGF18, Sprifermin, Merck) was shown to induce proliferation of chondrocytes. In a Phase II trial Sprifermin had increased total femorotibial joint cartilage thickness by 0.5 mm after 2 years in OA patients. There were no statistically significant differences in mean absolute change from baseline in total WOMAC pain scores for any sprifermin group, compared with placebo.

There are no treatments available that slow down or stop OA disease progression, have healing or beneficial biological activity on cartilage matrix production or re-balance the pathological shift of joint homeostasis and that at the same time influence OA pain.

As an efficacious disease modifying OA treatment, especially when associated with systemic or anatomical changes, would need to be given repeatedly and as a life-long treatment, it must be safe. High systemic drug exposures after systemic treatments would increase the risk of unwanted systemic effects or effects on cartilage, synovium and bone in non-diseased joints. Intraarticular injections, on the other hand, are not recommended more frequently than 6 times per year.

There is no compound available that has beneficial effects on structure and pain in OA and is efficacious with intermittent treatment.

R399E is the first therapeutic approach that has rapid and lasting effect on pain and at the same time halters disease progression by reducing cartilage destruction, inducing cartilage matrix production and normalizing joint homeostasis in OA animal models and human tissue with the same dose. The beneficial effect of R399E on pain in OA in vivo has been confirmed in 2 species (FIGS. 1 and 2). Also, the beneficial effect of R399E on cartilage structure in OA has been confirmed in 2 species (FIGS. 15, 16, 17). No other known molecule, protein or combinations of prior art show beneficial effect on pain and cartilage structure in OA, Furthermore R399E reduces inflammation and cytokine releases in relevant in vitro experiments using tissues and primary cells from healthy animals and human OA joint replacement surgeries that enables a normalization of joint homeostasis (FIGS. 8, 9, 10, 11, 13). R399E inhibits PGE2 release in relevant healthy animal and human OA in vivo experiments and furthermore the NGF induced production of PGE2 in OA meniscus cells (FIGS. 8, 12). PGE2 is an important mediator of cartilage degradation and pain (Lee et al. Gene. 2013 Sep. 25; 527(2):440-7). R399E shows anti-catabolic effects by preventing the release of GAG (Chun et al. Tissue Eng. Regen Med 2019 Jul. 5; 16(4):385-393) and release and reduced expression of ADAMTS5, MMP13 and MMP1 in in vitro experiments in human OA tissues and cell cultures and in healthy porcine cells (FIGS. 8, 13, 14). Metalloproteinases as ADAMTS5 and matrix metalloproteinases as MMP13 play an important role in the occurrence of OA (Bondeson et al. Clin Exp Rheumatol. 2008 January-February; 26(1):139-45 and Xie et al. Chem Med Chem. 2017 Aug. 8; 12(15):1157-1168). R399E shows pro-anabolic effects in healthy animal and human OA tissues and cell culture (FIGS. 18, 19, 20, 21, 22). R399E shows immunohistology and gene expression analysis the induction of Glycosaminoglycan and hydroxyproline synthesis and also the gene expression of Collagen-II, Collagen-VI, Sox9 and Aggrecan (FIG. 21). Treatment of chondrocyte cell culture of human OA with R399E shows an effect in cartilage formation also with infrequent administration of R399E.

The pro-anabolic effect of R399E is also shown by the up-regulation of biomarker assumed to display positive trend in OA as proC2, proC6 and CILP-2. This is shown for human OA tissue in FIG. 23.

R399E easily penetrates cartilage and can be found within the cartilage matrix close to the cells 7 days after IA injection. In an IA rabbit PK study, R399E was found in synovial fluid and cartilage, whereby 6 μg of R399E injected were detectable until day 3. Injections of 60 μg of R399E could be detected in synovial fluid for 14 days and in cartilage for up to 7 days. Despite the increased stability of the molecule compared to GDF5 wildtype, serum half-life did not exceed 3.20 hours and was quantifiable in minipigs and rabbits up to 72 hours and the safety profile was clean also after IA and IV application in rats, minipigs and rabbits in pharmacokinetic and nonclinical safety studies. Taken together with the low solubility of approximately 1 μg/mL in biological liquids at a physiological pH, we do not assume an increased safety risk by the increased stability.

Intermittent local (intraarticular) treatment with R399E is enough to have beneficial effects on pain and structure and results in low and very short systemic exposure. In contrast, to GDF5 wildtype, R399E is absorbed from surrounding liquid rapidly, if cartilage is present. This makes intraarticular treatment with R399E not only highly effective, but also safe.

R399E has rapid and lasting effect on pain in translational Osteoarthritis models with the same dose and regimen that results in beneficial effects on structure. Models were used in which the pharmacodynamic effect can be compared with that of drugs that are effective clinically for either pain (anti-NGF-antibody, Triamcinolone) or structure (Sprifermin).

### SUMMARY OF THE INVENTION

The preferred embodiment of the invention is the inject of R399E IA into joints of Osteoarthritis patients with and without joint inflammation to reduce inflammation and pain and to improve joint tissue structures.

Furthermore, R399E will reduce local cytokine and Prostaglandin E2 production and thereby reduce joint inflammation and pain. R399E will reduce local ADAMTS5 and MMP-13 production and thereby not only prevent cartilage cleavage, but also further reduce joint inflammation and pain by preventing DAMP release, which can also prevent DAMP sensitization of neurons. Reduced cytokine production may also restore responsiveness to endogenous BMPs and to the treatment itself by reducing the down-regulating effect on BMPR expression. R399E directly induces extra cellular matrix formation of osteoarthritic chondrocytes and can thereby support structural repair of osteoarthritic joints.

Inject R399E IA into joint after a traumatic event to prevent cartilage or meniscus degradation and to reduce inflammation. This will reduce the risk of later Osteoarthritis development.

The present invention is based on the finding of the inventors that it is possible by specific modifications in the region of the amino acid sequence of a GDF-5 related protein which is involved in the binding to BMPR-IB and/or BMPR-IA to change the protein in such a way that same has an improved ability of inducing cartilage formation and a reduced ability for inducing bone formation.

It was found out that proteins having an increased affinity for BMPR-IB and/or proteins having a reduced affinity for BMPR-IA are better capable for inducing cartilage formation while the formation of bone is reduced. These properties are especially pronounced in proteins showing both an increased affinity for BMPR-IB and a reduced affinity for BMPR-IA.

The GDF-5 related proteins of the present invention can be obtained by chemical modification or genetic engineering technology with recombinant proteins being preferred. The proteins can be obtained by replacing at least one amino acid residue relating to a BMPR-IB and/or BMPR-IA binding site in the amino acid sequence of a GDF-5 related protein.

A protein used for injection in patients suffering from OA or other inflammatory diseases is a variant of human GDF-5, whereby the arginine residue at position 399 is exchanged against glutamic acid (R399E). Referring to the mature sequence of GDF-5 this corresponds to a substitution at position 18. Surprisingly, it was found that this protein variant has a considerably reduced affinity for the BMPR-IA. In contrast, the affinity for the BMPR-IB is almost unaffected.

Preferably, the GDF-5 related protein (R399E) of the present invention is present as “isolated” proteins. This means that the protein of the present invention is substantially separated from other proteins and peptide molecules which are present in the natural source of the isolated protein (e.g. other polypeptides of the protein of the natural source). For example, a recombinant expressed peptide is considered isolated. According to a preferred embodiment of the invention, the variant protein is a recombinant protein. Further, a peptide is also considered isolated, if it has been altered by human intervention or expressed by an organism that is not its natural source. Moreover, an “isolated” protein is free from some of the other cellular material with which it is naturally associated or cell culture medium, when produced by recombinant techniques or chemical precursors or other chemicals when chemically synthesized. Specifically excluded from the definition of “isolated” protein, are unpurified mixtures or compositions.

Further subject matter of the present application is a pharmaceutical composition comprising the recombinant GDF-5 related protein or a nucleic acid or a vector or a host cell according to the invention. In principle, any pharmaceutical compositions which have already been published in context with GDF-5 related proteins are suitable. An expression vector or a host cell can be considered to be advantageous as active substances in a pharmaceutical composition. Also, combinations of a protein according to the invention with other proteins can be used in preferred pharmaceutical compositions. Of course, the invention also comprises pharmaceutical compositions containing further substances like e.g. pharmacologically acceptable additives or carriers. The formulation may include antioxidants, preservatives, colouring, flavouring and emulsifying agents, suspending agents, solvents, fillers, bulking agents, buffers, delivery vehicles, excipients and/or pharmaceutical adjuvants. For example, a suitable carrier or vehicle may be water for injection, physiological saline solution or a saline solution mixed with a suitable carrier protein such as serum albumin. A preferred antioxidant for the preparation of the composition of the present invention is ascorbic acid.

The solvent or diluent of the pharmaceutical composition may be either aqueous or non-aqueous and may contain other pharmaceutically acceptable excipients which are capable of modifying and/or maintaining a pH, osmolarity, viscosity, clarity, scale, sterility, stability, rate of dissolution or odor of the formulation. Similarly, other components may be included in the pharmaceutical composition according to the present invention in order to modify and/or maintain the rate of release of the pharmaceutically effective substance. Such modifying components are substances usually employed in the art in order to formulate dosages for parenteral administration in either unit or multi-dose form.

The finally formulated pharmaceutical composition prepared according to the present invention may be stored in sterile vials in form of a solution, suspension, gel, emulsion, solid or dehydrated or lyophilized powder. These formulations may be stored either in a ready-to-use form or in a form, e.g. in case of a lyophilized powder, which requires reconstitution prior to administration. The above and further suitable pharmaceutical formulations are known in the art and are described in, for example, Gus Remington's Pharmaceutical Sciences (18th Ed., Mack Publishing Co., Eastern, Pa., 1990, 1435-1712). Such formulations may influence the physical state, stability, rate of in vivo release and rate of in vivo clearance of the pharmaceutically effective component.

Other effective administration forms comprise parenteral slow-release, i.e. retarded, formulations, inhalant mists, or orally active formulations. For example, a slow-release formulation may comprise proteins bound to or incorporated into particulate preparations of polymeric compounds (such as polylactic acid, polyglycolic acid, etc.) or liposomes.

The pharmaceutical composition according to the present invention may also be formulated for parenteral administration, e.g., by infusion or injection, and may also include slow-release or sustained circulation formulations. Such parenterally administered therapeutic compositions are typically in the form of pyrogen-free, parenterally acceptable aqueous solutions comprising the pharmaceutically effective component(s) in a pharmaceutically acceptable carrier and/or diluent.

The pharmaceutical composition may comprise a matrix material, e.g. in cases where regeneration of cartilage is intended. It is advantageous to the protein, the nucleic acid, the expression vector or the host cell when they are applied in and/or on a biocompatible matrix material. Matrix material as used herein means a carrier or matrix acting as a scaffold for cell recruitment, attachment, proliferation and differentiation and/or as a potential delivery and storage device for the recombinant GDF-5 related proteins of the invention. In contrast to the solid matrices, carriers consist of amorphous materials having no defined surfaces and lacking a specific shape, i.e. alkyl cellulose, pluronics, gelatins, polyethylene glycols, dextrins, vegetable oils, sugars and other liquid and viscous substances.

Exemplary matrix materials are for example described in WO 98/21972. These matrix materials are equally suitable for the proteins according to the invention. The matrix material can be transplanted into the patient, e.g. surgically, wherein the protein or the DNA encoding the protein can be slowly released from the matrix material and then be effective over a long period of time. All types of matrix materials are useful in accordance with the present invention as long as they are biocompatible and selected for the intended area or indication of use. The matrix material can be a natural material, a modified natural material as well as a synthetic material. All already known matrices for morphogenetic proteins are encompassed. The extracellular matrix comprises for examples various collagens as for example types I, II, V, IX, X, XI and XIII, further proteoglycans and glycosaminoglycans as for example chondroitin sulfates, biglycans, decorines and/or hyaluronic acid or non-collagenous proteins as e.g. osteopontin, laminin, fibronectin, vitronectin and cartilage matrix protein. All mentioned natural materials may also be used in artificially modified forms. For a non-limiting list of useful carriers and matrices (see further Kirker-Head, 2000, Advanced Drug Delivery 43, 65-92).

A further possibility concerns liposomal formulations comprising the recombinant GDF-5 related protein according to the invention. Liposomes used in said formulations are commonly known to the person skilled in the art. In particular, preferred liposomal formulations are disclosed in WO 2008/049588. More preferred liposomal formulations are described on pages 9 to 13 of WO 2008/049588.

Furthermore, the GDF-5 variant protein (R399E) of the invention can be administered in combination with other pharmaceutically active substances. Said pharmaceutically active substances can be, for example, painkillers such as locally effective painkillers or other substances that have a positive effect on diseases, wherein the formation of cartilage is desired, like protease inhibitors. These are only examples of possible additives, and a worker skilled in the art can easily add other excipients which are in use in pharmaceutical preparations or which are generally regarded as safe.

Due to their improved capability of inducing cartilage formation, the recombinant GDF-5 variant proteins of the present invention are particularly suitable for use in the treatment of diseases, wherein the formation of cartilage is desired, but the formation of bone is undesirable.

Thus, another aspect of the present invention is the use of the present proteins (R399E), nucleic acids, vectors or host cells in the treatment of these diseases. In particular, the present protein, nucleic acids, vectors or host cells are for use in the treatment of cartilage defects or for the treatment of traumatic rupture or detachment of cartilage, in particular age-related cartilage defects for example due to wear, osteoarthritis, rheumatoid arthritis, sports diseases related injuries, like meniscus injury or ligament ruptures, disease which can affect the cartilage like chondrodystrophies, diseases characterized by disturbance of growth and subsequent ossification of cartilage, achondroplasia, costochondritis, spinal disc herniation and spinal disc repair, relapsing polychondritis, repair of cartilage defects associated with tumors, either benign or malignant, like chondroma or chondrosarcoma.

Another aspect is a method for the treatment of diseases, wherein the formation of cartilage is desired, but the formation of bone is undesirable comprising the step of administering a protein, nucleic acid, vector or host cell according to the invention to a patient in need of such treatment.

As used herein, the term “treating” refers to reversing, alleviating or inhibiting the progress of a disease, disorder or condition or one or more symptoms of such disease, disorder or condition to which such term applies. As used herein, treating may also refer to decreasing the probability or incidence of the occurrence of a disease, disorder or condition in a mammal as compared to an untreated control population or as compared to the same mammal prior to treatment. For example, as used herein, treating may refer to preventing a disease, disorder or condition and may include delaying or preventing the onset of a disease, disorder or condition or delaying or preventing the symptoms associated with a disease, disorder or condition. As used herein, treating may also refer to reducing the severity of a disease, disorder or condition or symptoms associated with such disease, disorder or condition prior to a mammal's affliction with the disease, disorder or condition. Such prevention or reduction of the severity of a disease, disorder or condition prior to affliction relates to the administration of the composition of the present invention as described herein to a subject that is not at the time of administration afflicted with the disease, disorder or condition. As used herein, treating may also refer to preventing the recurrence of a disease, disorder or condition or of one or more symptoms associated with such disease, disorder or condition.

### BRIEF DESCRIPTION OF THE FIGURES AND TABLES

FIG. 1 Pain Readout in Rat Osteoarthritis Model

Significant effect on pain within days in the late chronic phase of a surgical rat Osteoarthritis model.

FIG. 2 Pain Readout in Rat Osteoarthritis Model

In the rat ACLT+pMX OA model every 6 weeks IA treatment regimen is advantageous over every 4 or every 2 weeks treatment regimen when treatment is started in the early phase one week post-surgery (B).

FIG. 3 Pain Readout in Rabbit Osteoarthritis Model

Significant effect on pain in rabbit ACLT+pMx Osteoarthritis model 6 hours after the first IA injection with R399E

Baseline incapacitance measurement was made 2 times prior to surgery. OA was induced by ACLT+pMx knee surgery in rabbits in the right knee joint in week 0. R399E was injected intra-articularly (IA) in week 1 and static weight bearing was measured 6 hours later. Weight bearing was measured using a contact-free static incapacitance measurement whereby plates measure pressure put on the operated and injected right hindlimb in comparison to the left unoperated hindlimb. Data are percent weight put on the right over the left hindlimb, whereby 50% corresponds to equal loading of both legs and 0% to load on the unoperated left limb only.

FIG. 4 Pain Readout in Rabbit Osteoarthritis Model

Significant effect on pain in rabbit ACLT+pMx Osteoarthritis model 6 hours after first injection was confirmed in a second independent study and effects of one R399E injection were compared to clinically effective Triamcinolone

Baseline incapacitance measurement was made 2 times prior to surgery. OA was induced by ACLT+pMx knee surgery in rabbits in the right knee joint in week 0. R399E was injected intra-articularly (IA) in week 1 and static weight bearing was measured 6 hours later. Weight bearing was measured using a contact-free static incapacitance measurement whereby plates measure pressure put on the operated and injected right hindlimb in comparison to the left unoperated hindlimb. Data are percent weight put on the right over the left hindlimb, whereby 50% corresponds to equal loading of both legs and 0% to load on the unoperated left limb only. Effect size was compared to clinically effective Triamcinolone treatment. 1.41 mg Triamcinolone correspond to human equivalent dose calculation based on metabolic body weight, synovial fluid volume and cartilage surface area.

FIG. 5 Pain Readout in Rabbit Osteoarthritis Model

Effects of one R399E injection or Triamcinolone treatment on OA pain in a rabbit model of OA during the first 2 weeks after the first injection.

Baseline incapacitance measurement was made 2 times prior to surgery. OA was induced by ACLT+pMx knee surgery in rabbits in the right knee joint in week 0. R399E was injected intra-articularly (IA) in week 1 and 3 and static weight bearing was measured always before injections and 6 hours after the first injection. Weight bearing was measured using a contact-free static incapacitance measurement whereby plates measure pressure put on the operated and injected right hindlimb in comparison to the left unoperated hindlimb. Data are percent weight put on the right over the left hindlimb, whereby 50% corresponds to equal loading of both legs and 0% to load on the unoperated left limb only. Effect size was compared to clinically effective Triamcinolone treatment. 1.41 mg Triamcinolone correspond to human equivalent dose calculation based on metabolic body weight, synovial fluid volume and cartilage surface area. Trimacinolone.

FIG. 6 Pain Readout in Rabbit Osteoarthritis Model

Significant effect on OA pain of one IA R399E injection lasts at least 2 weeks until the next injection in a surgical OA model in rabbits. Also, in the chronic phase of the model, R399E has lasting significant effect on pain.

Baseline incapacitance measurement was made 2 times prior to surgery. OA was induced by ACLT+pMx knee surgery in rabbits in the right knee joint in week 0. R399E was injected intra-articularly (IA) in week 1, 3, 5, 7, 9 and 11 post surgery and static weight bearing was measured always before injections and 6 hours after the first injection. Weight bearing was measured using a contact-free static incapacitance measurement whereby plates measure pressure put on the operated and injected right hindlimb in comparison to the left unoperated hindlimb. Data are percent weight put on the right over the left hindlimb, whereby 50% corresponds to equal loading of both legs and 0% to load on the unoperated left limb only. All tested doses of R399E had significant beneficial effect on joint pain rapidly that lasted for at least 14 days until the next injection and until the end of the study.

FIG. 7 Pain Readout in Rabbit Osteoarthritis Model

The long-term effect of R399E on pain is comparable to the effect size of clinically effective anti-NGF-antibody treatment during the chronic phase in rabbit ACLT+pMx OA model

Baseline incapacitance measurement was made 2 times prior to surgery. OA was induced by ACLT+pMx knee surgery in rabbits in the right knee joint in week 0. R399E was injected intra-articularly (IA) in week 1, 3, 5, 7, 9 and 11 post surgery and static weight bearing was measured always before injections and 6 hours after the first injection. Weight bearing was measured using a contact-free static incapacitance measurement whereby plates measure pressure put on the operated and injected right hindlimb in comparison to the left unoperated hindlimb. Data are percent weight put on the right over the left hindlimb, whereby 50% corresponds to equal loading of both legs and 0% to load on the unoperated left limb only. All tested doses of R399E had significant beneficial effect on joint pain rapidly that lasted for at least 14 days until the next injection and until the end of the study. Effect sizes on chronic pain were compared to effect sizes reached with clinically effective anti-NGF-AB treatments in week 5 and 9 in the chronic phase of OA progression. Anti-NGF-AB was given twice to the same animals that had received Triamcinolone in week 1.

FIG. 8 Co-culture of Human Osteoarthritis Synovial Membrane and Cartilage Explants

In human Osteoarthritis synovium and cartilage co-cultures R399E reduces matrix GAG loss, Interleukin-1 and Prostaglandin 2 release.

FIG. 9 Human OA Chondrocyte 3D Alginate Bead Culture

Primary human OA chondrocytes (alginate bead culture, 380 mOsm, 300 ng/ml, 7 days) permanently treated with Lipopolysacharid (LPS), Interleukin-1 beta (IL1β), Tumor necrosis Factor-alpha (TNFa) or Interleukin-6 (IL6) show impaired Bone Morphogentic Protein Receptor (BMPR) expression. BMPR's are key for cartilage, bone and meniscus homeostasis. They are the main addressees of Bone Morphogentic Proteins like BMP2 or 7, but also of GDF5 and R399E.

AG-ALGIN-17-008: 5 donors human OA chondrocytes in monolayer, 48 h with IL1b 10 ng/mL, TNFa 10 ng/mL, IL6 100 ng/mL or LPS 1 μg/mL. Statistic: One-way ANOVA followed with Dunnet test (correction for multiple comparison). * Means statistically different with p<0.05.

FIG. 10 Porcine Meniscus Cultures

R399E decreases matrix loss and cytokine production in porcine meniscus cultures.

FIG. 11 Synoviocyte Cell line SW982 and Primary Human Osteoarthritis Synoviocyte Culture

IL-1 (A) and -6 (B) release in synoviocyte cell line SW982 and in primary OA synoviocytes (C, D)

SW982 (Synoviocyte cell line) cells were treated with three different concentrations of R399E for 72 hours. R399E significantly reduced IL-1β (A) and IL6 (B) levels at 300 ng/ml. Primary OA synoviocytes harvested from synovial membrane samples received from total knee replacement surgeries were treated with three different concentrations of R399E for 72 hours. R399E significantly reduced IL-1β (C) at 900 ng/ml and reduced IL6 levels (D).

FIG. 12 Primary Human Osteoarthritis Meniscus Cell Culture

R399E inhibits NGF stimulated PGE2 release in primary human meniscus cells in vitro.

FIG. 13 Primary Porcine Healthy Chondrocyte Culture

R399E inhibits IL-1beta stimulated upregulation of ADAMTS5 (A) expression and MMP1 (B) release in porcine chondrocytes.

FIG. 14 Primary Human Osteoarthritis Chondrocyte Culture

R399E reduces MMP13 and ADAMTS5 expression in human OA chondrocytes in 2 weeks alginate bead culture at 380 mOsm. Both proteases are major drivers of OA disease progression by cleaving collagen and aggrecan. The resulting DAMPs (Damage-associated molecular patterns) bind to pain (Rosenberg et al. Mol Cell Biochem. 2017 December; 436(1-2):59-69. doi: 10.1007/si 1010-017-3078-x. Epub 2017 Jun. 1) and inflammation mediating Toll-like receptors Miller et al. Arthritis Rheumatol. Author manuscript; available in PMC 2016 Nov. 1. Published in final edited form as: Arthritis Rheumatol. 2015 November; 67(11): 2933-2943. doi: 10.1002/art.39291.

FIG. 15 Structure Readout in Rabbit Osteoarthritis Model

R399E has significant beneficial effect on cartilage structure in a rabbit ACLT+pMx model of OA in histology (A) and micro-CT (B, C) read-outs. In micro-CT, cartilage thickness and volume where quantified by segmentation of the contrast enhanced joint cavity.

FIG. 16 Structure Readout in Sheep Osteoarthritis Model

R399E has significant beneficial effect on cartilage structure in a sheep joint instability pilot study of OA in histology. R399E was injected 3 times every 4 weeks starting 1 week post-surgery.

FIG. 17 Structure Readout in Sheep Osteoarthritis Model

R399E has beneficial effect (not statistically significant) on cartilage structure in a sheep joint instability pilot study of OA in MRI. R399E was injected 3 times every 4 weeks starting one week post-surgery.

FIG. 18 Primary Porcine Healthy Chondrocyte Culture

Extra cellular matrix production in Cartilage Tissue Analogue (CTA) 3D culture of porcine healthy chondrocytes at 380 mOsm with or without permanent R399E treatment shows significant pro-anabolic effect.

Primary porcine healthy chondrocytes (4 weeks 3D culture cartilage tissue analogue, 380 mOsm, 300 ng/ml).

FIG. 19 Primary Human Osteoarthritis Chondrocytes 3D Alginate Bead Culture

R399E dose-dependently increases extra cellular matrix production of human OA chondrocytes GAG, HPro, proC2.

FIG. 20 Primary Human Osteoarthritis Chondrocytes 3D Alginate Bead Culture

R399E dose-dependently increases Aggrecan production of human OA chondrocytes.

Effect of compounds on aggrecan production in human OA chondrocyte alginate beads. Chondrocytes were isolated from three independent donors. Cells were stimulated with different concentrations of the compounds over 7 days. Aggrecan was measured in the interterritorial matrix after bead depolymerization and compared to day 7 control levels.

FIG. 21 Primary Human Osteoarthritis Chondrocytes 3D Alginate Bead Culture

Extra cellular matrix production in two weeks alginate encapsulated 3D culture of human OA chondrocytes at 380 mOsm with or without R399E

Primary human OA chondrocytes (2 weeks alginate bead culture, 380 mOsm, 300 ng/ml)

FIG. 22 Primary Human Osteoarthritis Chondrocytes 3D Alginate Bead Culture

Intermitted treatment with R399E is enough to reach significant pro-anabolic effects over time. Human OA chondrocytes were cultured in alginate as previously described and treated one week, two weeks, three weeks or four weeks per months with R399E 300 ng/mL or left untreated and GAG content was quantified at the end.

Primary human OA chondrocytes (alginate bead culture, 380 mOsm, 300 ng/ml); Cells=number of chondrocytes, GAG=component of Aggrecan, Hpro (hydroxyproline)=component of Collagen type II, proC2=biomarker for Collagen type II production.

FIG. 23 Primary Human Osteoarthritis Chondrocytes 3D Alginate Bead Culture

Pro-anabolic biomarker measurement of proC2 (A), proC6 (B) and CILP-2 (C) in four weeks alginate encapsulated 3D culture of human OA chondrocytes at 380 mOsm with or without R399E.

Table 1 Treatment scheme and study outline of KK-rat-14-09

Three different regimen and 9 doses were tested in a rat ACLT+pMx model of OA. Study was powered with n=10 animals per group. Numbers in grey fields indicate doses applied intra-articularly (IA) in ng in 30 μl total volume injected. Gait analysis was performed every 2 weeks and vonFrey hypersensitivity testing in week 15 or 16.

Table 2 Pain Readout in Rat Osteoarthritis Model

Symptomatic benefit of different doses and regimen IA R399E treatment in a rat instability model of OA. The table lists only those effects that were >30% better than placebo. The most efficacious and sustainable effect was seen with 2 injections of 1350 ng every 6 weeks in rats.

### EXAMPLES

### Example 1

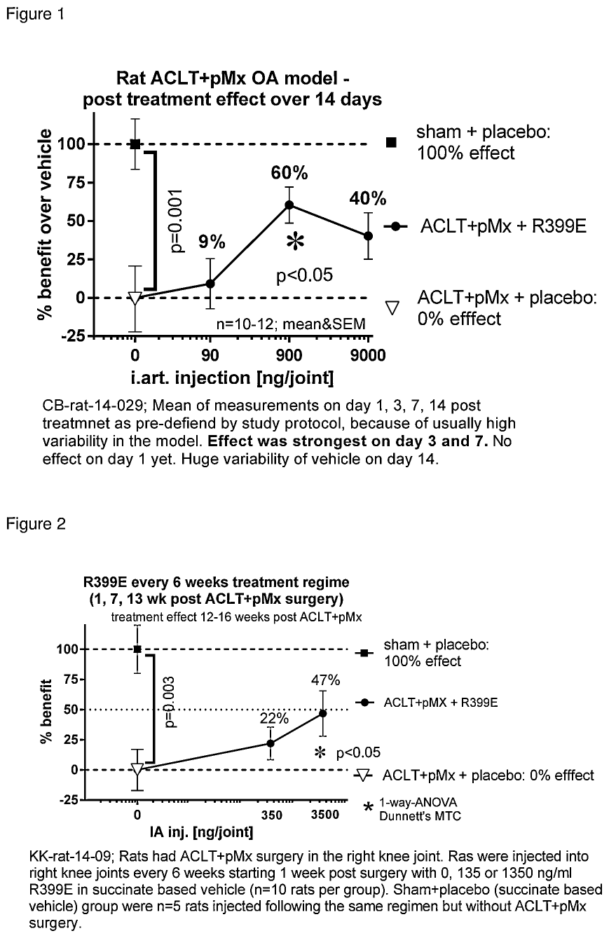

In a surgically induced chronic rat model of OA, one intraarticular (IA) injection of R399E has significant effect on pain within 14 days in late stage disease when given 12 weeks after the joint destabilization surgery (CB-rat-14-029, see FIG. 1).

The anterior cruciate ligament transection (ACLT) with resection of the medial meniscus (pMx) as an instability OA model in rodents was established in house as the changes in the joint are comparable to those found in OA patients (cartilage damage, osteophytes, subchondral sclerosis, impaired gait and inflammation-based hypersensitivity). Gait disturbance symptoms determined by the catwalk test were used as primary readout with an analogy to the clinical questionnaire asking patients to rate their pain experienced during walking on a flat surface (see Ferreira-Gomes et al. The journal of pain: official journal of the American Pain Society. 2008 October; 9(10):945-54. PubMed PMID: 18650131). ACLT+pMx surgery induced joint instability results in, punctate abnormal load on the medial tibial condyle that causes cartilage erosion already within a week (see Naveen et al. International journal of medical sciences. 2014; 11(1):97-105. PubMed PMID: 24396291. Pubmed Central PMCID: 3880996).

Gait disturbance typically occurs in the week after surgery (postsurgical pain) followed by a symptom free period and finally returns during the late chronic OA phase. This late gait disturbance phase is understood as OA pain phase. To investigate whether a single injection of R399E can produce a symptomatic benefit before structural reparation effects, in CB-rat-14-029, R3399E was administered as a single injection (at three doses IA) 12 weeks after ACLT+pMx surgery when gait disturbance due to chronic OA pain had been fully established. After habituation for 3 weeks to the test facility rats were either subjected to sham (skin incision) or ACLT+pMx surgery. To determine the OA-pain related symptoms gait disturbance was determined by the CatWalk test at 10, 11 and 12 weeks after surgery. All rats which showed gait disturbance symptoms were randomized into the 4 treatment groups (3 groups with different doses of R399E and placebo control) and received one IA injection at day 81 after ACLT+pMx surgery. Based on the gait parameter which was most sensitive to ACLT+pMx surgery during this 3-week period we identified 8 rats as asymptomatic and excluded them from the study. The remaining 40 rats were randomized on the basis of their gait disturbance into 4 groups receiving either IA placebo or R399E (90 ng, 900 ng or 9000 ng/joint) on day 80 after surgery. As pre-defined in the analysis plan, treatment effect was determined by the CatWalk test 1, 3, 7 and 14 days after this single injection and the mean over all 4 measurements was compared between groups. In this period six gait parameters have been identified to be significantly different between the sham+placebo und ACLT+pMx+placebo group and were therefore used to describe the OA disease related symptoms. We found that in this period immediately after the injection all these disease relevant gait parameters were positively affected by the injection of R399E. The percent benefit over placebo for all qualified parameters revealed that 900 ng/joint R399E was the most efficient dose producing a symptomatic benefit of 60%. The lowest dose [90 ng/joint] had almost no effect and the highest dose [9000 ng/joint] produced 40% benefit over placebo (see FIG. 1).

### Example 1.1

In the same rat model used in example 1, the effect of R399E on OA pain lasts for at least 6 weeks, until the next injection (KK-rat-14-009, see FIG. 2 and table 1 and 2). KK-rat-14-009 used the same surgical rat OA model and was designed to investigate whether chronic intra-articular (IA) injections of R399E have symptomatic benefit in a chronic rat osteoarthritis pain model when given constantly with different doses and regimen. Doses of 0.9-9000 ng monthly and the corresponding doses 135 ng, 1350 ng every 6th week and 45 ng and 450 ng every 2 weeks were chosen based on the in vitro EC50 of 108 ng/ml on cartilage matrix production (see FIG. 20) and a dose of 2000 ng every 2 weeks that was effective in rabbit pilot studies (data not shown) (see table 1). Rats were treated over 17 weeks post-surgery and symptoms were measured with the CatWalk gait analysis system and by the von-Frey hyperalgesia test. In the chronic disease course (week 12-16 after surgery), untreated rats develop symptoms which can be measured as gait impairment with the CatWalk system or mechanical hyperalgesia by the von-Frey test. According to a pre-defined analysis plan, we calculated means of catwalk measurements of week 12 to 16 and values higher than 30% over vehicle were regarded as relevant. The same 30%-benefit-over-vehicle limit was defined for the von-Frey measurement in week 16.

Analysis of the gait disturbance determined in the CatWalk test revealed that the every 2 weeks regimen with 45 ng resulted in a 16% benefit over the ACLT+pMx+vehicle group. 450 ng injected every 2 weeks reached 20% benefit which was still not statistically significant or meaningful. In the 4 weeks regimen 0.9 ng resulted in a relevant benefit over ACLT+pMx+vehicle of 52%, 9 ng had no effect of 4.4%, 90 ng displayed statistically significant effect of 82%, 900 ng had no effect of 1.4% over ACLT+pMx+vehicle and 9000 ng showed no meaningful effect of 17%. The corresponding dose in the every 6 weeks regimen of 135 ng/injection showed a trend of 22% benefit over ACLT+pMx+vehicle, while the higher dose of 1350 ng/injection had statistically significant and meaningful benefit of 47% over ACLT+pMx+vehicle (see FIG. 2 and Table 2).

In the von-Frey hyperalgesia test, injections every 2 weeks resulted in 104% reduced hypersensitivity compared to ACLT+pMx+vehicle with 45 ng, significant in 2-tailed t-test, and in 68% benefit with 450 ng. In the 4 weeks regimen, again, the 0.9 ng (71% benefit) and 90 ng (91% benefit) significantly reduced hypersensitivity, while 9 ng (−5%) and 9000 ng (−12%) had no effect. However, in the von-Frey test, also the 900 ng group reached 90% benefit over vehicle but no statistical significance. In the every 6 weeks regimen, the vehicle (vehicle) group showed less hypersensitivity with a higher variability than the vehicle groups that were treated more often and did not reach statistically significant difference to the sham group. 135 ng injections every 6 weeks had 16% benefit over vehicle and 1350 ng 70% benefit (see Table 2).

### Example 2

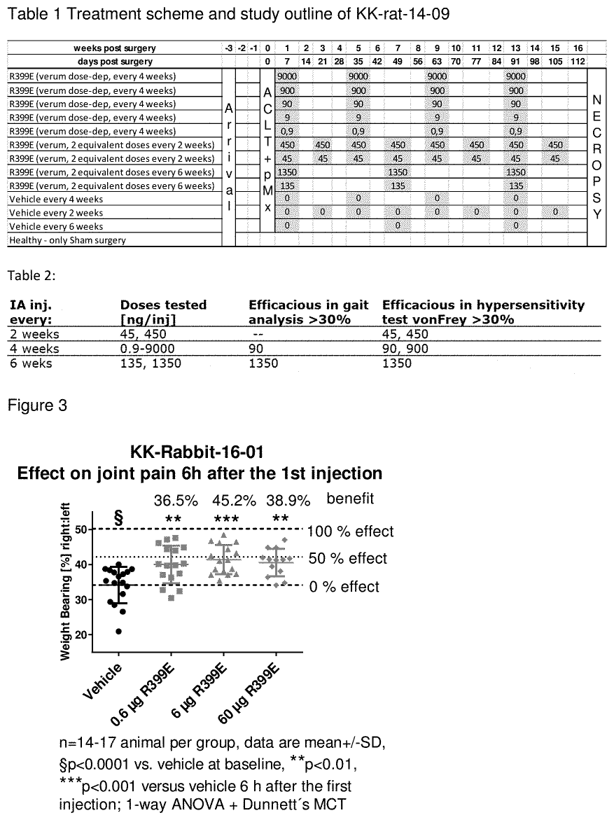

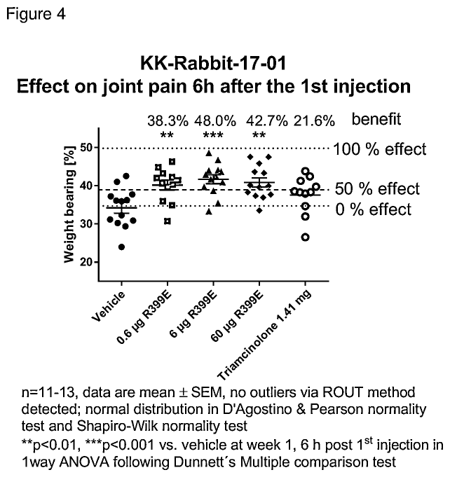

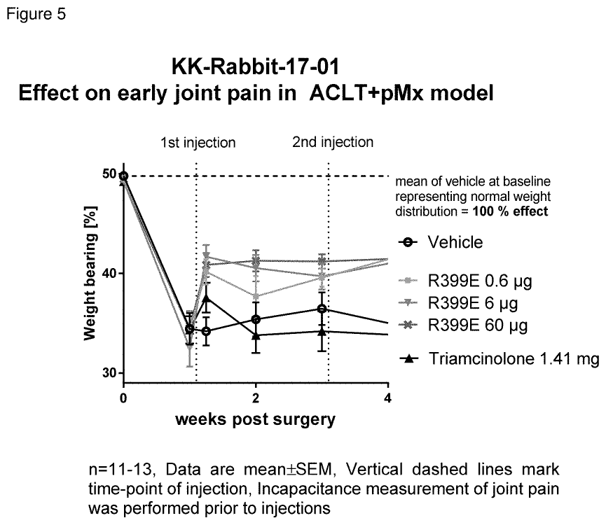

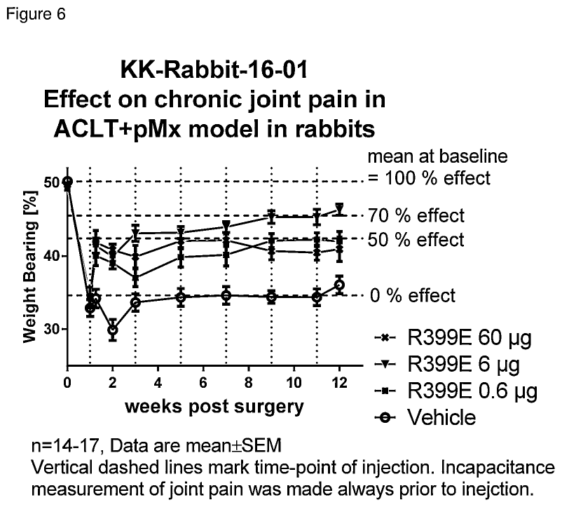

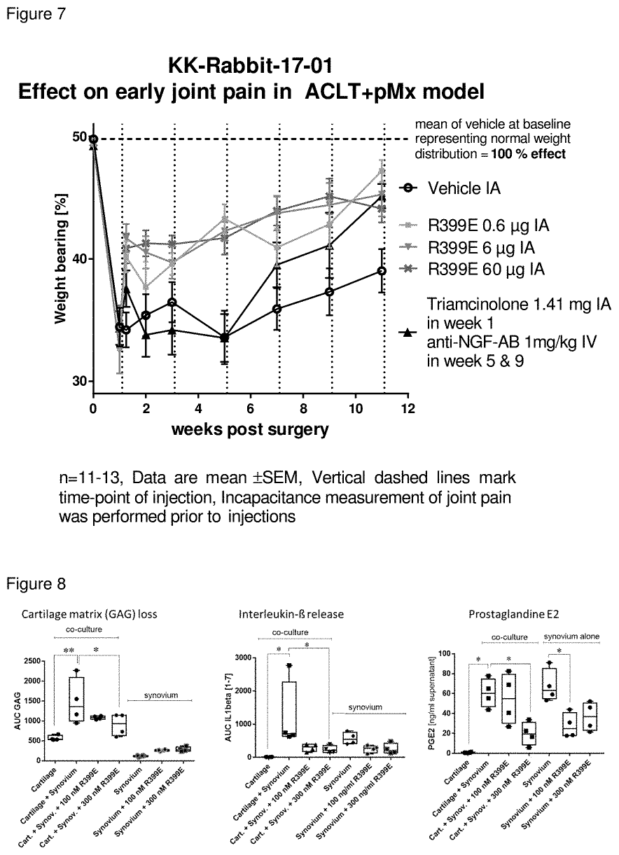

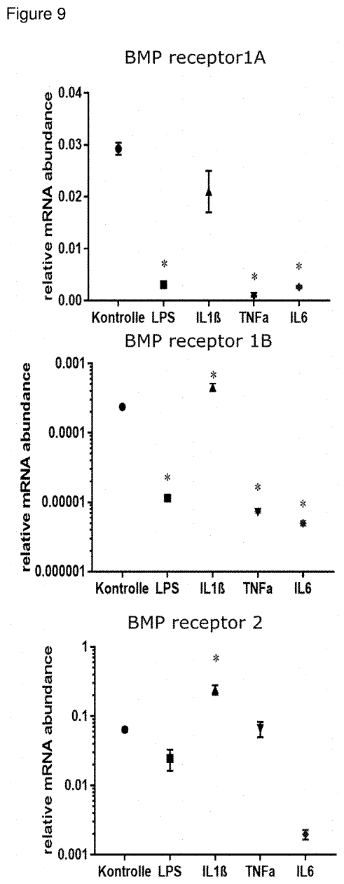

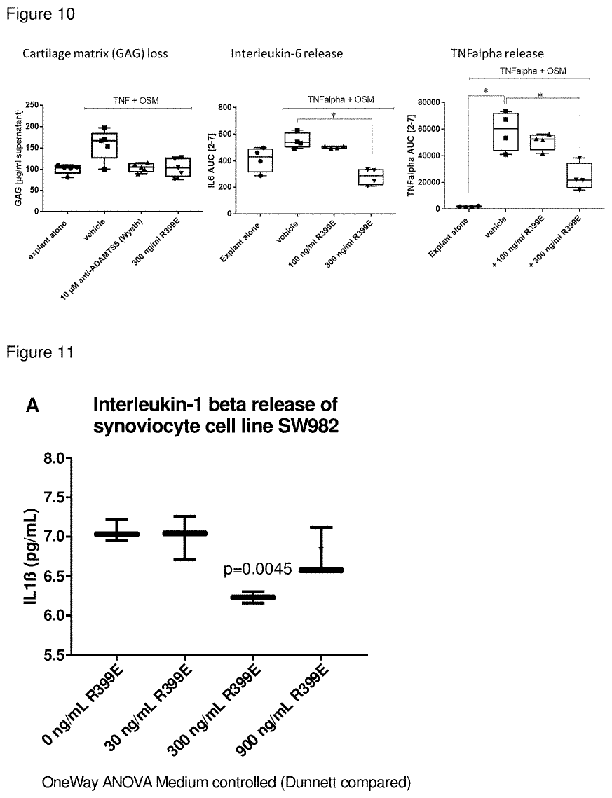

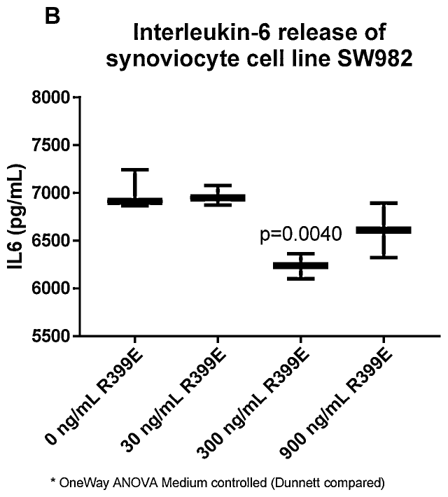

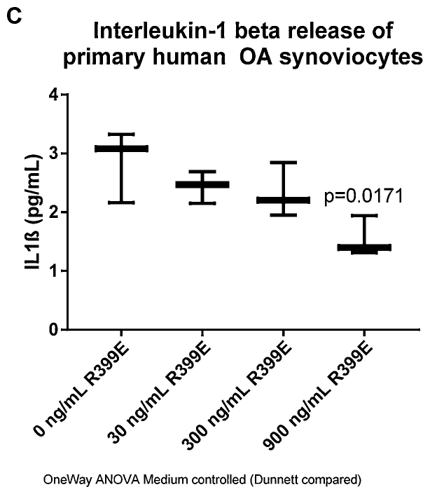

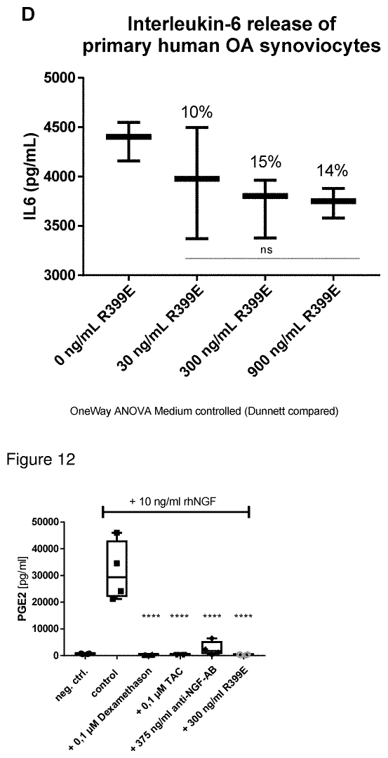

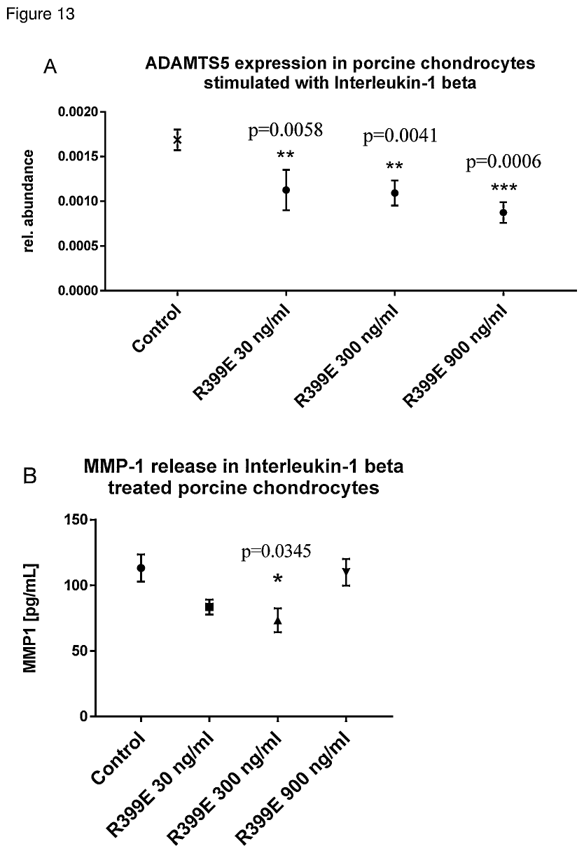

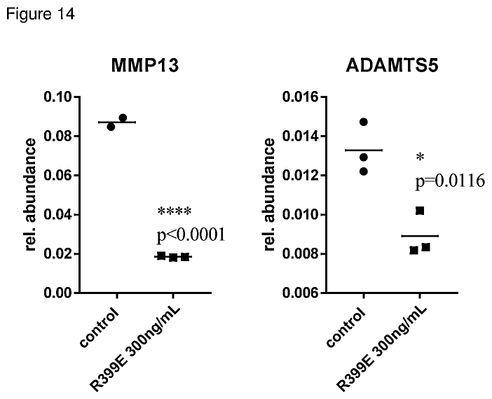

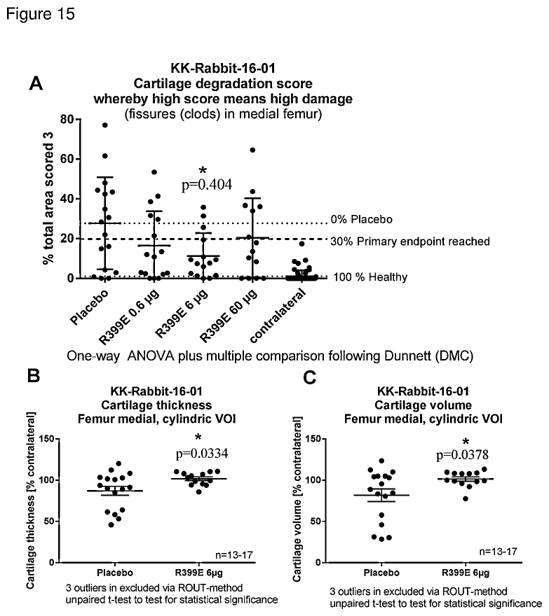

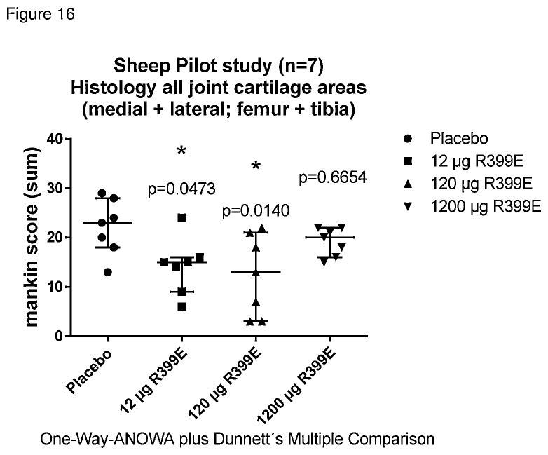

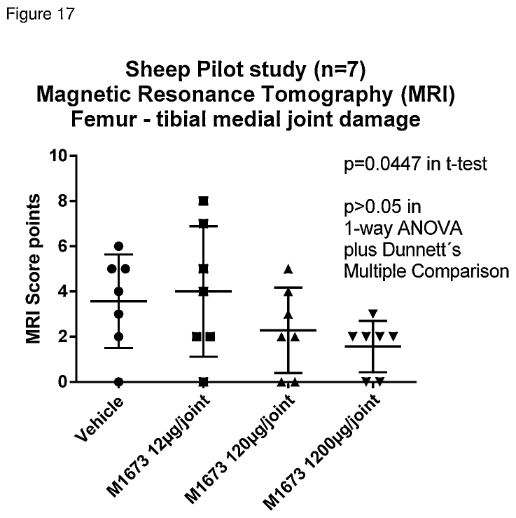

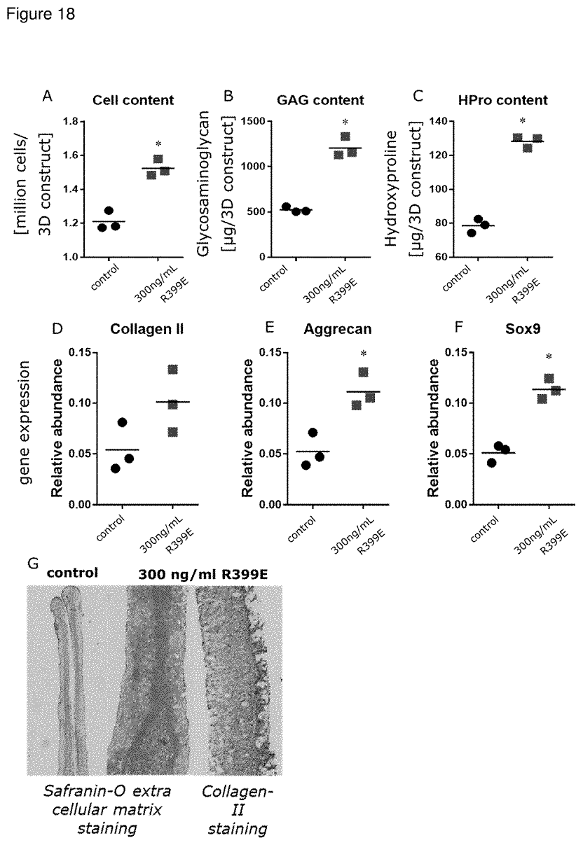

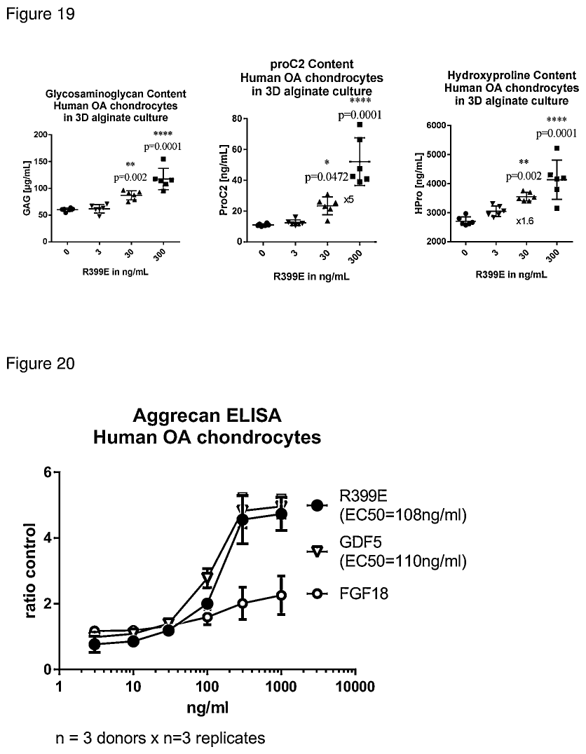

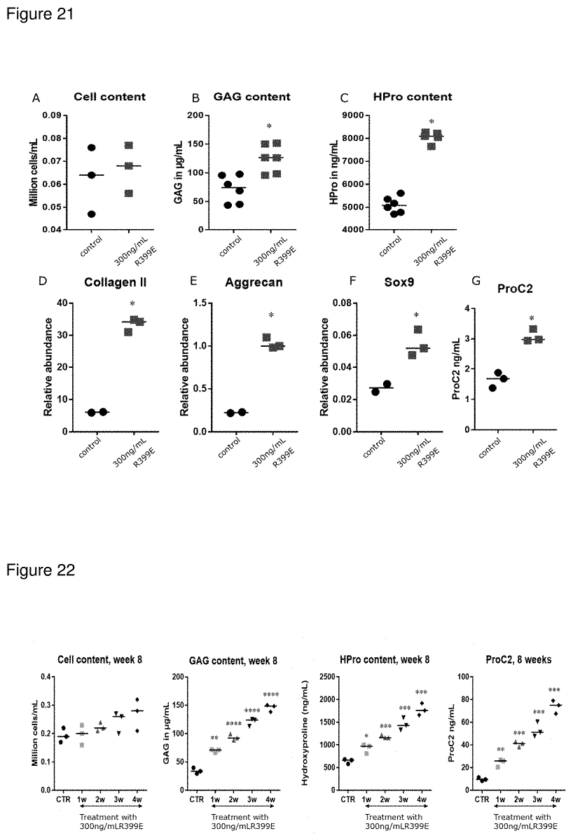

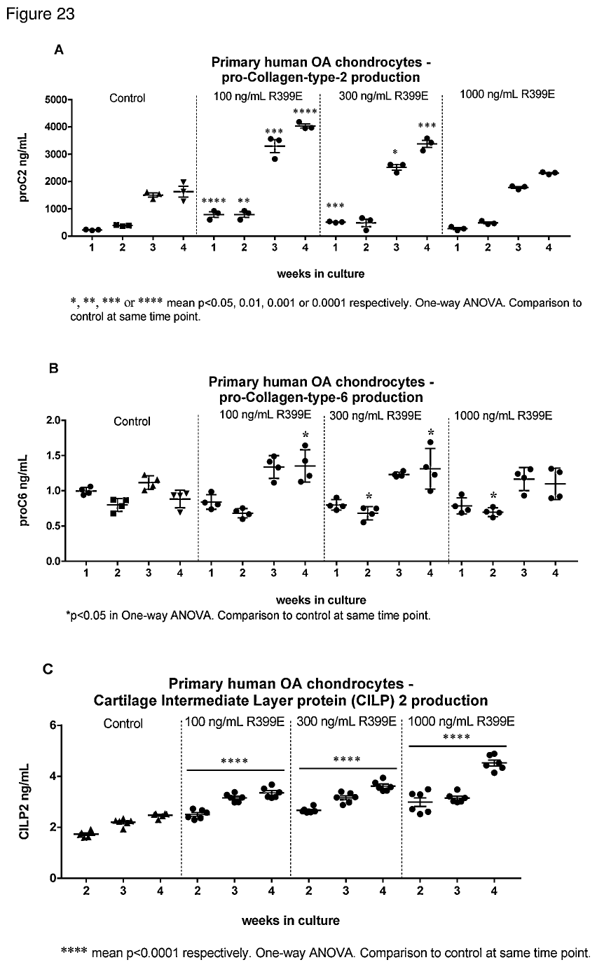

In a surgically induced rabbit model of OA, one intraarticular injection of R399E has significant effect on pain within 6 hours. This effect lasts for at least 2 weeks until the next injection. This result was confirmed in 2 independent studies (see FIGS. 3 and 4)

In KK-rabbit-16-01, anaesthetized rabbits were positioned on a warming pad and were slowly infused intravenously with 5% glucose solution during surgery. Right knee joints were shaved and disinfected. Skin, muscle and capsule were opened using scalpel and scissors. The patella was positioned laterally, and the fat pad incised to uncover the anterior cruciate ligament. The ligament was transected using a small clasp and a scalpel. The anterior horn of the meniscus was uncovered and detached from the menisco-tibial ligament. Under fixation using small forceps, the anterior half of the meniscus was resected. The joint was flushed with sterile physiological saline solution and the capsule and skin were closed in three layers using resorbable suture material. Rabbits were kept in cages until fully recovered from anesthesia and then brought back into the group. Rabbits were allowed to freely move and jump in a 56-m² stable until study end. For evaluation of joint loading an incapacitance test was performed. The weight bearing of each hind limb was measured via pressure plates and recorded electronically as ratio left unoperated to right operated knee joint as follows: right limb/(right limb+left limb)*100. The incapacitance device used was customized for well-trained group housed rabbits that are easy to handle and need no fixation to stand still. The rabbit was put onto the device and the hind limbs were positioned in the middle of the pressure measuring plates. The animal was not fixed or touched by an observer during the measurement to prevent an influence by the observer. The measurement was controlled by the connected PC and data were collected automatically and observer independently. Each measurement took approximately 5 seconds and was stopped manually when stable data over a minimum of 3 seconds were gained. In rare cases, when animals didn't stand still, the measurement was stopped and repeated afterwards.

ACLT+pMx surgery resulted in significant unloading of the right hind limb in week 1, 2, 3, 5, 7, 9, 11 and 12 post-surgery. Joint loading shifted from a 50:50% load of right compared to left hind limb to an approximate load of 66:34% left (un-operated) compared to right (operated) hind limb.

Animals were injected intraarticularly (IA) with placebo (R399E vehicle), 0.6, 6 or 60 μg R399E starting one week post-surgery and then 6 times in total every 14 days. Animals were euthanized in week 13, 2 weeks after the last injection.

Already 6 hours after the first injection (firsts measurement), all tested doses of R399E significantly restored loading of the right hind limb resulting in load ratios of approximately 60:40 left:right, meaning 36.5% benefit with 0.6 μg (p=0.0023), 45.2% benefit with 6 μg (p=0.0002) and 38.9% benefit with 60 μg (p=0.0016) (see FIG. 3).

### Example 3

In a surgically induced rabbit model of OA, the onset of R399E's effect on pain after one intraarticular injection is as immediate as with clinically effective Triamcinolone in the acute and inflammatory early phase 1 week after the surgery. The effect of R399E on pain reaches statistical significance with all doses, while Triamcinolone effects are slightly lower (FIG. 4).

In KK-rabbit-17-01, the same surgically induced OA model and study design was used as in KK-rabbit-16-01 aiming to compare effects of R399E with effect sizes of clinically effective Triamcinolone in the early and acute phase one week post-surgery.

As described above, osteoarthritis-like cartilage degradation was induced experimentally by transection of the anterior cruciate ligament (ACLT) and partial anterior resection of the medial meniscus (pMx) in 62 female 36-37 weeks old New Zealand white (NZW) rabbits. Animals were randomly distributed by bodyweight to five groups. Four groups received 0 μg (vehicle control, n=13), 0.6 μg (n=12), 6 μg (n=13) and 60 μg (n=13) R399E in 200 μl vehicle per injection. An additional, experimental fifth group (n=11) was injected with Triamcinolone (1×IA injection in week 1 post-surgery) to compare the pharmacological effect of R399E with Triamcinolone which has demonstrated symptomatic efficacy in OA-patients. Intra-articular (IA) treatment started one week post-surgery with the first injection.

Triamcinolone and all tested doses, 0.6 μg (38.3% over vehicle, p<0.01), 6 μg (48% over vehicle, p<0.001) and 60 μg (42.7% over vehicle, p<0.01) had significant effect on pain already 6 hours after the first injection (see FIG. 4).

### Example 4

The pain-relieving effect of 6 and 60 μg R399E injected IA into operated knees during the acute early phase post-surgery in a rabbit OA model lasts at least 2 weeks until the next injection, while the effect of 1.41 mg Triamcinolone has gone away already 1 week after the first injection (FIG. 5).

Like described above, in KK-rabbit-17-01, Osteoarthritis-like cartilage degradation was induced experimentally by transection of the anterior cruciate ligament (ACLT) and partial anterior resection of the medial meniscus (pMx) in female rabbits.

In week 2 post surgery, 6 μg (35.7%, p=0.0802) and 60 μg (40.8%, p=0.0417) R399E accounted for symptomatic effect >30% compared to placebo, whereby the symptomatic effect of Triamcinolone (−11.6%, p=0.89) was completely gone and animals showed even more relieving posture than placebo treated animals at that time point (see FIG. 5).

### Example 5

R399E IA injections have beneficial effect on pain also during the chronic phase of a surgically induced OA model in rabbits (FIG. 6).

Throughout the whole experiment KK-rabbit-16-01 described above, the medium dose of 6 μg had the highest observed mean effect over time and exerted 49% benefit in week 2, 57% in week 3, 55% in week 5, 60% in week 7, 69% in week 9 and 11 and 72% week 12 (all time points p=0.0001). The 0.6 μg (p=0.0027) and 60 μg (p=0.0001) groups had reached their maximum effect levels of approximately 40% over vehicle between week 5 and 7. This effect size was then stable until study end, while the effect of 0.6 μg (˜50% benefit) was slightly higher than that with 60 μg (˜40%) at later time points beginning in week 9 (see FIG. 6)

### Example 6

In a surgically induced rabbit model of OA, the size of R399E's effect on pain after one intraarticular injection is comparable to that of clinically effective anti-NGF-antibody treatment during the chronic phase of disease progression (FIG. 7).

Like in example 5 (time course of KK-rabbit-16-01) in KK-rabbit-17-01, the symptomatic benefit of R399E IA treatment sustained throughout the whole study. Injections started one week after surgery and were then made every other week for six times in total. Groups 1-3 in that study were treated with R399E IA, and group 4 with placebo IA. In group 5, as described above, Triamcinolone was injected IA in week 1. The same animals received clinically effective anti-NGF-antibody IV at human equivalent doses in weeks 5 and 9 after the Triamcinolone effect had completely gone away. In addition, placebo IA injections were given in weeks 3, 5, 7, 9 and 11 to that group. Observer independent contact free incapacitance measurement was performed like in the examples before 6 hours after the first injection and then every other week always prior to injections.

IV treatment with 1 mg/kg anti-NGF-antibody resulted in up to 56% effect on pain (p=0.0195), an effect size that is comparable to the 47.1% (p=0.0426), 57.8% (p=0.0091) and 75.7% (p=0.0011) effect on pain that were achieved with the 3 doses of R399E at the same time point (see FIG. 7)

### Example 7

In human synovium plus cartilage explants cultures, R399E reduces matrix loss (GAG release) and thereby contributes to significant normalization of joint homeostasis. In addition, R399E reduces the release of cytokines that are responsible for pain and inflammation in OA and impair the joint homeostasis: IL1 and PGE2 (FIG. 8). These cytokines are not only directly inducing pain and inflammation in OA patients, but they also down-regulate BMP receptor expression in chondrocytes. The resulting reduced responsiveness of chondrocytes to BMPs may accelerate the disease progression (FIG. 9).

The effect of R399E on matrix modulation (GAG release), PGE2 and pro-inflammatory cytokines were investigated in a human OA co-culture model, comprised of cartilage explants plus synovial membrane, or synovial membrane alone. The tissues were co-cultured for 7 days with sampling after 1, 4, 7 days. Co-cultivation of OA cartilage explant with OA synovial membrane, significantly induced GAG release into the supernatant. Treatment with R399E (100 and 300 ng/mL) inhibited GAG release, reaching statistical significance with 300 ng/mL (p=0.016). Cultivation of synovial membrane alone did not induce GAG release, indicating that the OA cartilage is the main source of GAG in the co-culture system (FIG. 8).

Co-cultivation of OA cartilage together with OA synovium robustly induced release of IL1β and PGE2 into the supernatant. R399E inhibited IL1β (p=0.0245 with 100 ng/mL and p=0.0159 with 300 ng/mL, see FIG. 11) and PGE2 (p=0.9872 with 100 ng/mL and p=0.0057 with 300 ng/mL) production in the co-culture system. Cultivation of synovial membrane alone resulted in significantly more PGE2 in the supernatant than cultivation of explant alone (p=0.0001), indicating that the synovial membrane is the major source of PGE2 in this system. Both R399E doses tested inhibited the unstimulated PGE2 release by the synovium (p=0.007 with 100 ng/mL and p=0.027 with 300 ng/mL).

In summary, R399E inhibited matrix degradation in a co-culture of human OA cartilage and synovial membrane. In addition, R399E interfered with autocrine and/or paracrine signaling between OA cartilage and OA synovium, represented as an inhibition of inflammatory cytokines and pain mediating PGE2.

### Example 8

In meniscus tissue cultures stimulated with TNFalpha plus Oncostatin, R399E reduced the release of such cytokines that are responsible for pain and inflammation in OA and impair the joint homeostasis (TNFa, IL6) and it prevents matrix loss (GAG, FIG. 10).

Porcine meniscus was cultivated with the aim to investigate the effect of R399E on the release of prostaglandin E2 (PGE2) and the cytokines IL1β, IL6, IL8 and TNFα. Full-slices of porcine meniscus (meniscus explants) were stimulated with T+O (20 ng/mL TNFα+10 ng/mL OSM (Oncostatin A)). In addition, meniscus explants were treated with different concentrations of R399E (300, 900, 1200 ng/mL). Overall incubation time was 7 days with sampling after 2, 5 and 7 days. As a control, meniscus explants were stimulated, but untreated (T+O) or unstimulated (explants alone). Stimulation with T+O induced IL6 from porcine meniscus. Treatment with R399E inhibited T+O induced IL6 release (p=0.0001 with 900 ng/mL research batch, p<0.015 with 300 and 1200 ng/mL tox batch, p<0.005 with 300 and 900 ng/mL GMP batch). R399E inhibited T+O induced PGE2 release up to 44% with the GMP batch, up to 72% with the research batch and 49% with the GLP batch, though reaching no statistical significance with p<0.05. No significant difference between batches was observed. The effect of R399E on ILβ, II-8 and TNF-alpha release was inconsistent and not dose-dependent in this experimental setting (see FIG. 10).

In summary, the data indicate that the meniscus contributes to the inflammatory environment of knee OA and that treatment with R399E reduces concentrations of pro-inflammatory cytokines and PGE2 coming from the meniscus which can result in pain relief after IA injection in OA animal models and in OA patients.

### Example 9

Also, in a synoviocyte cell line (SW982) and in primary human Osteoarthritis synoviocyte cell cultures R399E reduces cytokine release of interleukin-6 (FIG. 11 A, C) and -1 (FIG. 11 B, D) and thereby helps to normalize joint homeostasis and prevent pain, inflammation and further disease progression (FIG. 11)

### Example 10

In primary human meniscus cell cultures stimulated with nerve growth factor (NGF), R399E reduces the release of PGE2, responsible for pain and inflammation in OA. Primary meniscus cells from total knee replacement surgeries were freshly prepared one day post-surgery within the frame of an ethical permission. First, skin and muscle were removed to isolate the menisci. The menisci were transferred into a 10 cm dish filled with HAM's F12+1% P/S+1% Amphotericin B. The tissue was cut into small pieces of approximately 3×3 mm and transferred into a sterile beaker for digestion. Digestion was performed for 16 h in 50 ml of a 0.4% Collagenase in HAM's F12+1% P/S+1% Amphotericin solution at 37° C., 7.5% CO2 under constant stirring. After 16 h the solution was pipetted through a 100 μm filter followed by a 40 μm filter and then centrifuged for 5 min at 1400 g. The remaining pellet, containing the cells was resuspended in 20 ml of HAM's F12+1% P/S+1% Amphotericin+10% FCS. Cell number was counted, and cell viability was determined. Finally, 10,000 cells per well were seeded in a 96 well plate. Cells were cultivated up to 1 week to reach confluency. In between medium was exchanged once. When cells reached confluency, they were stimulated with 10 ng/ml rhNGF and/or treated with 0.1 μM Dexamethasone, 0.1 μM Triamcinolone, 375 ng/ml anti-betaNGF antibody, 300 ng/ml R399E or left untreated (negative control). The effect of the individual compounds was compared to cells which were stimulated with rhNGF only. After 2 days incubation, the supernatant was removed for determination of Prostaglandin E2 (PGE2) in the medium. Treatment of primary meniscus cells with rhNGF induced PGE2, which was significantly inhibited with all tested compounds. The effect of R399E compares to that of clinically effective Dexamethasone, Triamcinolone and anti-NGF-antibody treatments (FIG. 12).

### Example 11

R399E reduces ADAMTS5 (A disintegrin and metalloproteinase with thrombospondin motifs 5) expression and Matrix metalloproteinase (MMP) release in porcine healthy chondrocytes (FIG. 13) and in human OA chondrocytes (FIG. 14). ADAMTS5 is a catabolic protease that is responsible for pathological cleavage of cartilage matrix during OA progression. R399E thereby prevents further cartilage destruction and reduces the production of damage-associated-molecular-pattern-molecules (DAMPs) such as endogenous DNA and other cartilage matrix breakdown products. These molecules are relevant in accelerating OA pathology and are responsible for OA related inflammation and pain and sensitization of neurons.

Porcine chondrocytes were isolated from the femoral heads of pigs, approximately 1 years of age obtained from a local slaughterhouse (Arras, Reichelsheim-Beerfurth). To remove cells from soft tissues, cartilage was digested sequentially with 0.25% w/v collagenase (Serva GmbH, Cat. No. 17465), in HAM's F12 (Gibco®, Life Technologies, Cat. No. 21765) for 45 mins at room temperature and 0.1% w/v collagenase in HAM's F12 with 1% penicillin/streptomycin (Gibco®, Life Technologies) overnight at 37° C. The resulting cell suspension was filtered through 100 μm, then 40 μm cell strainers (Becton Dickinson GmbH), washed several times by centrifugation and resuspended in culture medium. Freshly isolated porcine chondrocytes were first cultured 7 days in monolayer in DMEMHG, 10% Fetal Calf Serum (FCS, Promocell GmbH), 50 μg/mL Ascorbate-2-phosphate and 0.4 mM Prolin and then cultured at 15 000 cells/well for qPCR analysis or 200 000 cells/well for MMP1 measurement in a 24 well plate in the same medium with addition of 10 ng/ml IL-1β and treated with R399E at 30, 300 and 900 ng/mL or left untreated for 3 (MMP1) or 7 days (qPCR).

For gene expression, RNA was isolated using the RNeasy Mini Kit from Qiagen. mRNA concentration and quality were analyzed with an Agilent Bioanalyser with an Agilent RNA 6000 Nano Chip from Agilent technologies Inc. The reverse transcription was realized with the SuperScript III First-Strand Synthesis SuperMix from Invitrogen Corp and followed by a RNAse H treatment. qPCR was performed with the SYBR Green JumpStart Taq Ready Mix from Sigma using primers for porcine ADAMTS5 (‘TCACACTGCTCATGACGAAA; GCAAGTGTGTGGACAAAACC). 60S ribosomal protein L13a (RPL13A) was used as a house-keeping gene.

For MMP1 measurement, supernatants were collected and MMP1 was measured using the Human MMP3-Plex Ultra sensitive Kit (MSD).

Human chondrocytes were isolated from the cartilage harvested from three OA patients who underwent a total knee or hip replacement. All patients signed an informed consent. Cell isolation consisted in a 45 minutes digestion with collagenase 0.25% (1/10 dilution of collagenase NBG4 from Serva 2.5% in HAM's F12). The loosened cells were discarded, and the cartilage further digested overnight with collagenase 0.1% (1/25 dilution of collagenase NBG4 2.5% in HAM's F12 with 1% Penicillin/Streptomycin) to extract the chondrocytes.

Freshly isolated human OA chondrocytes were first cultured 5 days in monolayer in DMEM High glucose with 10% FBS, 0.4 mM proline and 50 μg/mL ascorbate-2-phosphate, 1% Penicillin/Streptomycin and then cultured at 200 000 cells/well in a 24 well plate in the same medium and treated with R399E at 30, 300 and 1 000 ng/mL or left untreated for 7 days. For gene expression, RNA with the RNeasy Mini Kit from Qiagen. mRNA concentration and quality were analyzed with an Agilent Bioanalyser with an Agilent RNA 6000 Nano Chip from Agilent technologies Inc. The reverse transcription was realized with the SuperScript III First-Strand Synthesis SuperMix from Invitrogen Corp and followed by a RNAse H treatment. qPCR was performed with the Taqman Universal PCR Mastermix from Life Technologies with the corresponding TaqMan Gene expression assay from Applied Biosystems. EF1alpha was used as a house-keeping gene.

### Example 12

In a surgically induced rabbit instability OA model (ACLT+pMx, KK-rabbit-16-01) 0.6 (p=0.205) and 6 μg (p=0.0404) had meaningful >30% beneficial effect on cartilage morphology in gross morphological analysis (FIG. 15A). Micro-CT based quantification of cartilage volume in the 6 μg group (best results in gross morphology) revealed significantly higher cartilage volume and thickness compared to the placebo group (FIGS. 15B and C).

Like described in example 2, the anterior cruciate ligament (ACLT) was transected and approximately half of the medial meniscus was resected (pMx) in female rabbits Animals were injected intraarticularly (IA) with placebo (R399E vehicle), 0.6, 6 or 60 μg R399E starting one week post-surgery and then 6 times in total every 14 days. Animals were euthanized in week 13, 2 weeks after the last injection.

For micro-CT analysis knee joints were separated in femur and tibia during necropsy process. Afterwards they were fixed in 4% paraformaldehyde (PFA, Merck, Darmstadt, Germany; in 1× phosphate buffered saline (PBS) pH 7.4, Gibco, Thermo Fisher Scientific, Waltham, USA) for at least 5 days to reach full fixation. Before micro-CT image acquisition, joints were rinsed in 1×PBS to wash PFA residues and were individually packed into small plastic shot glasses (2cl, EDEKA GUT&GÜNSTIG, Germany) filled with Moltofill™ elastic (Akzo Nobel Deco GmbH, Cologne, Germany) free of air bubbles. Specimens were scanned using a micro-CT system (SkyScan 1176; Bruker, Kontich, Belgium) with an X-ray source of 65 kV/384 μA, a pixel size of 17.60 μm and a 1 mm aluminum filter. After scanning, cross-sectional slices were generated with NRecon software (Bruker). Each scan was reconstructed using defined threshold values to distinguish bone & Moltofill™ (same radiocontrast like bone) from the negative contrast of cartilage with beam hardening and ring artifact corrections applied. All datasets were adjusted to anatomical markers with DataViewer software (Bruker) in the same manner to ensure uniform analysis. Three-dimensional analysis was performed using CTAn software (Bruker). The volume of interest (VOI) was applied on the weight bearing regions of the medial femoral condyles, with a dimension of 3502.8 μm in diameter (199 pixels). Inside this VOI, cartilage volume and cartilage thickness were calculated of the right and left (contralateral) medial femoral condyles and expressed as % values of corresponding contralateral joints (FIG. 15 B, C).

For gross morphological investigation, the articular surfaces of tibia and femur were stained in toluidine blue (0.05%) for 30 s at room temperature followed by dipping in demineralized water and an air-drying period of 15-20 minutes. In order to increase the contrast between smooth and fibrillated tissue the stained surfaces were dipped for 1 s in black ink (Higgins black India ink (Chartpak Inc, Leeds, Mass., USA) followed by awaiting period of 3 s and 3 s tap water rinsing. After an additional air-drying period of 15 minutes the surfaces were imaged using a Discovery V12 macroscope (Carl Zeiss Microscopy GmbH, Jena, Germany) and photographed with an Axiocam HRC camera and appropriate software AxioVision 4.8.2 (Carl Zeiss Microscopy, Jena, Germany). The magnification was chosen in a way that the entire articular surface is filling the imaging format. The motorized optical system was used for reconstruction of 3D images from acquired Z-staples. Height of the Z-staple was determined manually by scrolling through the region of interest in each joint. 10-20 single images were acquired in a Z-stack and combined to the final image for the Cavaleri-analysis.

Total joint surface areas were measured using image analysis software and a score was used to quantify morphological changes. The total joint surface areas were increased by surgery. This finding is expected and consistent with other studies using this model and also with other surgical models and different species. R399E had no effect on this parameter. In the gross-morphology sum score, means of all three treatment groups, independent from dosing had improved approximately 30%, considering that an improvement of 100% would correspond to the contralateral mean level, and 0% to the vehicle mean level. Areas with only mild changes, that were rated with a score of 1, were more in R399E treated groups than in vehicle treated animals. Again, all three doses reached a meaningful effect of 30% improvement over the mean of the vehicle group. In areas with a score of 2, standing for the amount of medium damaged cartilage, the mean of the 0.6 μg group showed a 30% benefit compared to the mean of the vehicle group. The most distinct structural effect was seen when looking at the amount of severe damaged cartilage with fissures (score 3). 0.6 μg R399E resulted in approximately 40% less areas with a score of 3, 6 μg reduced areas with fissures with an effect size of 50%, reaching statistical significance (p=0.0404) and 60 μg exerted 30% improved mean value compared to the mean of the vehicle group, reaching no statistical significance (FIG. 15A).

### Example 13

In a surgically induced sheep model of OA, 3 intraarticular injections of R399E given every 4 weeks resulted in significantly improved histological scores (FIG. 16) and in better MRI scores (FIG. 17) in tendency compared to placebo already in a pilot study with 7 animals per group only.

In this experiment, the medial meniscus transection model (Cake, 2013 Osteoarthritis and Cartilage 2013; 21: 226-236) was used to induce Osteoarthritis (OA) like changes, with an ‘in life’ phase of 12 weeks. The test item R399E was administered intraarticularly (IA) in a monthly regimen at 3 different doses (12, 120 and 1200 μg/joint) starting at day 7 after surgery. The primary outcome measures of the study were: Structural improvements on medial and lateral femoral condyle cartilage determined by quantitative scoring of histological sections. R399E improved this outcome significantly. Here, R399E was most efficacious with its medium dose of 120 μg/joint.

Osteochondral samples (6×6 mm) were collected from the load-bearing cartilage regions of the lateral and medial femoral condyles and lateral and medial proximal tibial condyles. Each sample was obtained from the central portion of the joint, determined using measurement of each joint. A ruler was used to mark the midpoint of the condyle and the midpoint was used as the center of the osteochondral sample. Samples were fixed in 10% buffered saline and decalcified in 10% EDTA solution for 4 weeks followed by one week in 5% formic acid. Paraffin-embedded sections (5 □m thickness) prepared. Sections were stained with Toluidine Blue and Safranin O-Fast Green to highlight structure and cartilage (Schmitz et al 2010 Osteoarthrtis and Cartilage. 18 S3 S113-116). The modified Mankin score was used to quantify the histological changes in the cartilage.

Sections were obtained from the four compartments of the operated joint and scored using a modified Mankin score. When the histological scores were added together, there was a statistically significant reduction in damage in animals receiving 12, 120 μg/joint R399E compared to vehicle controls (see FIG. 16). Sub-analysis of the different components of the Mankin score showed that the reduction in damage was not due to any one measured parameter but that the reduction was spread across all parameters.

Magnetic Resonance Imaging (MRI) was obtained from each operated limb post mortem using a low field MRI (Esoate). The MR images were scored blindly by a European Imaging Specialist using a modified sMOAKS score (refer to Moya-Angeler, 2016 March; 23(2):214-20. doi: 10.1016/j.knee.2015.11.017. Epub 2016 Jan. 27).

For MRI, the joint was considered as 3 units—medial and lateral femoro-tibial and femoro-patella joints. For each region of the joint the following were scored: Articular cartilage loss, Osteophytes, Joint effusion, Bone marrow lesions (subchondral bone hyperintensity).

MRI were obtained of all operated limbs post-mortem using a low-field magnet. The MR images were scored blindly by a European Imaging Specialist using a modified sMOAKS score. When the scores for all 3 compartments of the complete joint or in the medial femoral-tibial compartment alone were analyzed, there was a trend in sMOAK score in animals treated with 120 μg and 1200 μg R399E/joint compared to controls (see FIG. 17).

### Example 14

In porcine healthy chondrocyte cell culture experiments, R399E significantly increases cartilage extra cellular matrix production and cell proliferation (FIG. 18).

Porcine chondrocytes were isolated and cultured in a scaffold-free 3D culture system (Cartilage Tissue Analogue, CTA) as described elsewhere (Gigout et al., 2017 Osteoarthritis and cartilage, 25:1858-1867). One million cells/3D construct were seeded in DMEM High Glucose supplemented with 10% FCS, 0.4 mM Proline and 50 μg/mL ascorbate-2-phosphate and treated with R399E 300 ng/mL or left untreated for four weeks with N=3.

At the end of the culture, the 3D cell constructs were collected to evaluate their DNA, GAG and hydroxyprolin content or gene expression by qPCR. For each condition, samples were also fixed and embedded in paraffin for histology (N=3). Prior to GAG, hydroxyprolin and DNA measurement, the 3D cell constructs were digested with papain (overnight with papain 0.125 mg/mL, L-Cystein 5 mM in Papain buffer, 60° C.). The DNA was measured with the QuaniT PicoGreen ds DNA kit from Invitrogen according to the recommendation of the manufacturer. The GAG was quantified with the dimethylmethylene blue (DMMB) assay (Farndale et al., 1986 Biochem Biophys Acta 883:173-177) and HPro by HPLC as described in Gigout et al., 2007.

For gene expression, RNA was isolated with the RNeasy Mini Kit from Qiagen. mRNA concentration and quality were analyzed with an Agilent Bioanalyser with an Agilent RNA 6000 Nano Chip from Agilent technologies Inc. The reverse transcription was realized with the SuperScript III First-Strand Synthesis SuperMix from Invitrogen Corp and followed by a RNAse H treatment. qPCR was performed with the Sybr-Green Jumpstart Taq Ready Mix (Sigma-Aldrich) with 200 nM of the reverse and forward primers. EF1alpha was used as a house-keeping gene.

For histological evaluation, the 3D cell constructs were fixed with 4% paraformaldehyde 30 minutes at room temperature, washed three times in PBS and stained for extra cellular matrix with Safranin-O or for Collagen-type-2.

### Example 15

In human OA chondrocyte cell culture experiments, permanent exposure to R399E significantly and dose-dependently increases Glycosaminoglycans (GAG), Hydroxyproline (HPro) and pro-collagen-type-2 (proC2) production (FIG. 19).

Human chondrocytes were isolated from the cartilage harvested from three OA patients who underwent a total knee or hip replacement. All patients signed an informed consent. Cell isolation consisted in a 45 minutes digestion with collagenase 0.25% (1/10 dilution of collagenase NBG4 from Serva 2.5% in HAM's F12). The loosened cells were discarded, and the cartilage further digested overnight with collagenase 0.1% (1/25 dilution of collagenase NBG4 2.5% in HAM's F12 with 1% Penicillin/Streptomycin) to extract the chondrocytes. Each condition was performed with N=4.

Freshly isolated human OA chondrocytes were first cultured 5 days in monolayer in DMEM High glucose with 10% FBS, 0.4 mM proline and 50 μg/mL ascorbate-2-phosphate, 1% Penicillin/Streptomycin and adjusted at 380 mOsm (osmolarity confirmed with an Osmometer). The cells were then harvested and 2×106 cells were resuspended in an alginate solution (1.25% alginate from Fluka in 0.2 M HEPES from AppliChem and 1.5 M NaCl from Merck, adjusted to pH 7.4) and the cell suspension was poured drop by drop in 120 mM CaCl2 (Merck) containing 10 mM HEPES (AppliChem). The cell drops polymerized for 15 minutes under agitation to form alginate beads and were washed three times with a 150 mM NaCl solution. The alginate beads were first cultured for seven days without treatment in culture medium adjusted to 380 mOsm. Subsequently, the beads were transferred into 24 well ultra-low binding plates (VWR) with 5 beads/well in one mL of culture medium at 380 mOsm supplemented with 300 ng/mL R399E, or 12.5 μM HCL (control). After 14 days, the alginate beads were dissolved for one hour in 460 μL of 55 mM Na-citrate (Merck) with 150 mM NaCl at pH 8 and 40 μL of 2.5% collagenase. Next, 500 μL of DMEM high glucose or PBS were added and the solution centrifuged: GAG, HPro and ProC2 were measured in the dissolved alginate supernatant. GAG and HPro were analyzed as described above. ProC2 was measured as described in Gudmann et al., 2014 Int J Mol Sci, 15:18789-18803

### Example 16

In human OA chondrocyte cell culture experiments, permanent exposure to R399E significantly and dose-dependently increases Aggrecan protein levels with an EC50 of 108 ng/mL (FIG. 20).

Cartilage biopsies were isolated during total knee replacement surgery from 3 human donors and minced and digested. Cells were cultured until and frozen at P1 in liquid nitrogen (LN). Cells were thawed from LN and brought in culture at a cell density of 10000 cells/cm2 and cells were grown till confluency. 8 days later, cells confluent at P2 were trypsinized and counted and beads were made (day −5). After 5 days of culture (day 0), beads were stimulated three times for 7 days (on day 0, 2 and 4). After 1 week the beads were harvested and analyzed for aggrecan content.

Included controls are day 0 beads in regular culture medium and day 7 beads with vehicle control medium (1:50 dilution of 10 mM HCl pH0.2 in regular growth medium).

The aggrecan content of the samples was determined using the commercially available PG-ELISA (cat #KAP1461) from Diasource. The experimental OD-values were subtracted with the blanco control. Absolute amounts of aggrecan were calculated based on standard curve equation. Ratio's compared to day 7 beads (no stimulation-vehicle control medium only) were calculated and compared. 4PL fitting of the average ratios of 3 donors was used to calculate the EC50 values.

### Example 17

In human OA chondrocyte cell culture experiments, permanent exposure to R399E significantly increases hyaline cartilage matrix production over time (FIG. 21).

Human OA chondrocytes were isolated, cultured and embedded in alginate as described above and treated with 300 ng/mL R399E or left untreated. After dissolution of the beads, the cells were content with a ViCell Cell analyzed from Beckman Coulter. The GAG, HPro and ProC2 were measured in dissolved alginate as described above. Gene expression analysis was performed on the cells as described above.

### Example 18

Intermitted treatment with R399E is enough to reach significant pro-anabolic effects over time. Human OA chondrocytes were cultured in alginate as previously described and treated one week, two weeks, three weeks or four weeks per months with R399E 300 ng/mL or left untreated. After eight weeks (two months), the cell, GAG, HPro and ProC2 contents were evaluated significantly (FIG. 22).

Example 18 In human OA chondrocyte cell culture experiments, permanent exposure to R399E significantly increases pro-anabolic Biomarker production of proC2, proC6 and CILP-2 (FIG. 23).

human OA chondrocytes were cultured in alginate as previously described and treated with R399E 300 ng/mL for four weeks or left untreated. ProC2, Proc6 and CILP2 were measured in the culture medium at different time points. ProC2 was measured as mentioned above, Proc6 was measured by Nordic Bioscience and CILP2 was measured with the ELISA kit abx151073 from Abbexa.

1. A method for the treatment of cartilage defects and pain in a patient comprising administering to said patient a GDF-5 mutant protein with the amino acid exchange R399E.

2. The method according to claim 1, wherein the cartilage defects are selected from osteoarthritis, rheumatoid arthritis, sports related injuries like meniscus injury or ligament ruptures, and diseases which can affect the cartilage like chondrodystrophies, diseases characterized by disturbance of growth and subsequent ossification of cartilage, achondroplasia, costochondritis, spinal disc herniation and spinal disc repair, relapsing polychondritis, repair of cartilage defects associated with tumors, either benign or malignant, like chondroma or chondrosarcoma and pain.

3. A method for the prevention of cartilage or meniscus degradation by reducing inflammation and pain in a patient comprising administering to said patient a GDF-5 mutant protein with the amino acid exchange R399E.

4. The method according to claim 1, wherein said GDF-5 mutant protein with the amino acid exchange R399E is administered by injection in the affected joins intraarticular.

5. A pharmacological composition for the treatment of cartilage defects and pain comprising a GDF-5 mutant protein with the amino acid exchange R399E and at least one other pharmacological effective ingredient.

6. The pharmacological composition according to claim 5, wherein said other pharmacological effective ingredient is Sprifermin.

7. The pharmacological composition of claim 4, further comprising one or more acceptable additives or carriers.

8. The pharmacological composition of claim 5, further comprising one or more acceptable additives or carriers.

9. The method according to claim 1, wherein the cartilage defect is osteoarthritis.

10. A method according to claim 9, wherein the GDF-5 mutant protein with the amino acid exchange R399E is injected into joints of said patient, with or without joint inflammation, to reduce inflammation and pain and to improve joint tissue structures.
